(19) United States
(12) Patent Application Publication
(10) Pub. No.: US 2005/0238577 A1
(43) Pub. Date: Oct. 27, 2005

(54) Isolation of epithelial cells or their biochemical contents from excreta after in vivo isotopic labeling

(75) Inventor: Marc K. Hellerstein, Kensington, CA (US)

Correspondence Address: MORRISON & FOERSTER LLP, 425 MARKET STREET, SAN FRANCISCO, CA, 94105-2482, US

(73) Assignee: The Regents of the University of California, Oakland, CA (US)

(21) Appl. No.: 11/094,387

(22) Filed: Mar. 29, 2005

Related U.S. Application Data
(60) Provisional application No. 60/557,734, filed on Mar. 29, 2004.

(51) Int. Cl. A61K 51/00; A61K 49/00

(52) U.S. Cl. 424/1.11; 424/9.1

(57) ABSTRACT

The methods of the present invention allow for the non-invasive or minimally invasive isolation of epithelial cells or their biochemical contents from excreta after in vivo isotopic labeling. Once isolated, the labeled epithelial cells or their labeled biochemical contents find use as kinetic biomarkers of diseases of epithelial tissues including epithelial cancers and epithelial proliferative disorders such as psoriasis and benign prostate hyperplasia. The biomarkers are useful in diagnosing a disease of epithelial tissue origin, monitoring therapeutic efficacy of compounds or combinations of compounds used to treat diseases of epithelial tissue origin, screen new chemical entities or biological factors or combinations of chemical entities or biological factors, or mixtures thereof, for therapeutic activity in animal models of diseases of epithelial tissue origin or in human clinical trials, or test for toxicity on epithelial tissues from exposure to therapeutic compounds or new chemical entities or environmental chemicals such as industrial or occupational chemicals, environmental pollutants, food additives, and cosmetics.

### CROSS REFERENCE TO RELATED APPLICATION

This application claims priority to U.S. provisional application No. 60/557,734 filed on Mar. 29, 2004, which is hereby incorporated by reference in its entirety.

### FIELD OF THE INVENTION

The invention relates to methods for measuring changes in biochemical processes that may underlie various diseases and disorders. More specifically, the invention relates to measuring the molecular flux rates of these biochemical processes for diagnostic, prognostic, and therapeutic purposes.

### BACKGROUND OF THE INVENTION

Epithelial cancers, derived from epithelial tissues, are the most common and medically important oncologic disorders in the Western world. The carcinogenic process in epithelial tissues, as in other tissues, is characterized by an extremely long pre-cancer phase. Indeed, carcinogenesis is believed to be a multi-step process that evolves over decades, starting with a genetic damage event (initiation) and proceeding through a series of events (promotional phase) that culminates in a clinically apparent neoplastic growth. The proliferation of initiated cells—i.e., the process of mitotic cell division, involving DNA replication—is a critical component of the promotional phase of epithelial carcinogenesis. For this reason, many therapeutic strategies to prevent epithelial cancers that have been proposed and tested are based on the goal of reducing proliferation of cells in the target epithelial tissue (i.e., reducing mitogenesis). The target cells here are initiated but normal (i.e., pre-cancer) cells in a tissue.

Epithelial tissues, by definition, communicate topologically with the body space outside of the body of a metazoan organism. This communication may take the form of a luminal surface (i.e., a tubular structure with epithelial cell lining) or an integumental surface (e.g., skin, mucosa of the lip or tongue, etc.). The cells of an epithelial tissue are therefore either directly or indirectly exposed to, and in contact with, the outside environment. This feature has allowed development of certain clinically non-invasive (or minimally invasive) diagnostic procedures for disorders involving selected epithelial tissues. Examples of such procedures include cervical swabs (pap smears), fecal occult blood testing (hemoccult), urine cytology, sputum cytology, and breast nipple aspirate cytology for cancer screening or diagnosis.

At present, however, there are few clinical diagnostic methods available for reliably monitoring either the natural evolution of epithelial carcinogenesis or for evaluating the efficacy of interventions such as, anti-proliferative agents, aimed at reducing cancer risk. As noted above, the topologic communication of epithelial tissues with the outside space has allowed for the development of useful cytologic or other techniques for identifying relatively late-stage cancers. For the most part, however, these diagnostic modalities are not useful as biomarkers in the long pre-cancer phase. Because the pre-cancer phase is the optimal period for cancer prevention, current diagnostic tests are not of value for preventative efforts.

The absence of biomarkers of cancer chemoprevention efficacy has been noted by numerous authorities to be one of the leading problems and highest priorities in the field of cancer prevention. Accordingly, it would be extremely valuable to have generally applicable non-invasive methods for evaluating risk of cancer or other diseases of epithelial tissues and for monitoring therapeutic interventions in epithelial tissues. Such methods are disclosed herein.

### SUMMARY OF THE INVENTION

The present invention provides methods for characterizing and evaluating the progression of diseases of epithelial tissue origin, evaluating or characterizing the therapeutic efficacy of compounds or combinations of compounds or mixtures thereof (e.g., chemical entities, drug candidates, drug leads, already-approved drugs, biological factors and combinations and mixtures thereof) on diseases or conditions of epithelial origin, evaluating and characterizing toxic effects on tissues of epithelial origin from exposures to compounds or combinations of compounds or mixtures of compounds, and evaluating and characterizing toxic effects on tissues of epithelial origin from exposures to environmental chemicals including industrial or occupational chemicals, environmental contaminants or pollutants, food additives, and cosmetics.

In one embodiment, the method allows for the measurement of changes in one or more biomarkers of a disease of epithelial tissue origin by: 1) exposing a subject to one or more compounds; 2) administering an isotope-labeled substrate (i.e., labeled metabolic precursor) to the subject for a period of time sufficient for the labeled metabolic precursor to enter into one or more metabolic pathways of interest and thereby enter into and label at least one target molecule of interest within the metabolic pathway of interest in the subject; 3) obtaining one or more samples from the subject, wherein the one or more samples comprise at least one-isotope labeled targeted molecule of interest and are obtained from one or more body spaces external to the subject; 4) measuring the content, rate of incorporation and/or pattern or rate of change in content and/or pattern of isotope labeling of the targeted molecule or molecules of interest; 5) calculating molecular flux rates in the one or more metabolic pathways of interest based on the content and/or pattern or rate of change of content and/or pattern of isotopic labeling in the targeted molecule or molecules of interest; 6) measuring the molecular flux rates in the one or more metabolic pathways of interest according to steps 2) through 4) in a control subject not administered the one or more compounds; and 7) comparing the molecular flux rates in the one or more metabolic pathways of interest in the subject administered the one or more compounds to the molecular flux rates in the one or more metabolic pathways of interest in the subject not administered the one or more compounds.

In one embodiment, the collection of the sample is by non-invasive or minimally invasive means for example, by the collection of ductal fluid by lavage, skin scrapings, skin flakings, seminal fluid, nasal secretions, stool (feces), tears, sweat, flatulence, and the like, and epithelial cells and/or their biochemical components are isolated from the collected samples and further analyzed as described supra and infra.

In another embodiment, the molecular flux rates being measured in the one or more metabolic pathways of interest are relevant to an underlying molecular pathogenesis, or causation of, one or more diseases of epithelial tissue origin.

In a further aspect, the molecular flux rates in the one or more metabolic pathways of interest are known to or thought to contribute to the initiation, progression, severity, pathology, aggressiveness, grade, activity, disability, mortality, morbidity, disease sub-classification or other underlying pathogenic or pathologic feature of the one or more diseases of epithelial tissue origin.

In yet another aspect, the molecular flux rates in the metabolic pathways of interest contribute to the prognosis, survival, morbidity, mortality, stage, therapeutic response, symptomology, disability or other clinical factor of the one or more diseases of epithelial tissue origin.

In another embodiment, the molecular flux rates of said one or more metabolic pathways of interest are measured concurrently.

In a further embodiment, the concurrent measurement of the molecular flux rates from said metabolic pathways of interest is achieved by use of stable isotopic labeling techniques and the isotope label used is a stable (i.e., non-radioactive) isotope. In one aspect, the stable isotope used in the stable isotopic labeling is stable isotope-labeled water. In a particular aspect, the stable isotope-labeled water is ²H₂O.

In another embodiment, the concurrent measurement of the molecular flux rates from said metabolic pathways of interest is achieved by use of radioisotope labeling techniques.

In one aspect of the present invention, the one or more compounds is an already-approved drug. In a further aspect, the already-approved drug is a Federal Food and Drug Administration-approved drug.

In one aspect, the already-approved drug is selected randomly. In another aspect, the already-approved drug is selected on the basis of a specific biochemical rationale or hypothesis concerning a hypothesized role in the molecular pathogenesis of one or more diseases of epithelial tissue origin.

In yet another embodiment of the present invention, the one or more compounds is a new chemical entity. In another aspect, the one or more compounds is a biological factor.

In another embodiment of the present invention, one or more animal models of epithelial tissue disease are used for evaluating the actions on molecular flux rates in one or more metabolic pathways potentially related to disease in a subject. In a further embodiment, the one or more animal models of epithelial tissue disease is chosen from psoriasis, skin photoaging, skin rashes, breast cancer, prostate cancer, colon cancer, pancreatic cancer, and lung cancer.

In yet another aspect of the present invention, the one or more metabolic pathways of interest are measured in response to a specific dose or a range of doses of said one or more compounds.

In still yet another aspect of the invention, the one or more metabolic pathways of interest are chosen from breast epithelial cell proliferation, colon epithelial cell proliferation, prostate epithelial cell proliferation, ovarian epithelial cell proliferation, endometrial cell proliferation, bronchial epithelial cell proliferation, pancreatic epithelial cell proliferation, keratin synthesis in skin, and keratinocyte proliferation.

In a further aspect of the present invention, the already-approved drug is screened for actions on multiple biochemical processes concurrently.

In one embodiment of the present invention, the subject is chosen from rabbits, dogs, mice, rats, guinea pigs, pigs non-human primates, and humans. In a further aspect of the invention, the subject is a human.

In another embodiment of the invention, the isotope labeled substrate is chosen from ²H₂O, ²H-glucose, ²H-labeled amino acids, ²H-labeled organic molecules, ¹³C-labeled organic molecules, ¹³CO₂, ¹⁵N-labeled organic molecules, ³H₂O, ³H-labeled glucose, ³H-labeled amino acids, ³H-labeled organic molecules, ¹⁴C-labeled organic molecules, and ¹⁴CO₂. In a further embodiment, the isotope labeled substrate is ²H₂O.

In yet another embodiment of the invention, the one or more compounds are administered according to established or hypothesized dose ranges that have the potential for biological activity in said subject.

In still another embodiment of the invention, the one or more samples obtained from one or more spaces external to the subject are collected at known times or intervals after administration or contacting said subject to said isotope-labeled substrate and after exposing said subject to said one or more compound. In yet a further embodiment of the invention, the one or more spaces external to the subject is chosen from the urethra of the penis, vagina, uterus, gastrointestinal tract, respiratory tract, buccal cavity, skin surface, and breast duct.

In another aspect, the one or more samples collected from the one or more spaces external to the subject is chosen from urine, semen, vaginal secretions, stool, gastrointestinal secretion, sputum, skin flakes, and breast fluid.

In another embodiment, combinations of two or more compounds are exposed to the subject.

In yet another embodiment of the invention, synergistic, complementary, or antagonistic actions of combinations of compounds on molecular flux rates through the one or more metabolic pathways are determined by comparing said molecular flux rates in said subject exposed to the combination of compounds to said molecular flux rates in said subject exposed to a single compound alone or not exposed to any of said compounds being tested.

In one aspect, the combinations of compounds are selected randomly. In another aspect, the combinations of compounds are selected on the basis of a specific biochemical rationale or hypothesis concerning a hypothesized role of one or more of said compounds in the molecular pathogenesis of said one or more of diseases of epithelial tissue origin.

In another embodiment of the present invention, a method for evaluating an effect on one or more biomarkers of toxicity of epithelial tissue is provided, the method comprising: 1) exposing a subject to one or more compounds; 2) administering an isotope-labeled substrate to the subject for a period of time sufficient for the isotope-labeled substrate to enter into one or more metabolic pathways of interest and thereby enter into and label one or more targeted molecules of interest within the one or more metabolic pathways of interest in the subject wherein the one or more metabolic pathways of interest are related to one or more toxic effects; 3) obtaining one or more samples from the subject, wherein the one or more samples comprise one or more isotope-labeled targeted molecules of interest and are obtained from one or more external spaces to the subject; 4) measuring the content, rate of incorporation and/or pattern or rate of change in content and/or pattern of isotope labeling of the targeted molecule or molecules of interest; 5) calculating molecular flux rates in the one or more metabolic pathways of interest based on the content and/or pattern or rate of change of content and/or pattern of isotopic labeling in the molecule or molecules of interest; 6) measuring the molecular flux rates in the one or more metabolic pathways of interest according to steps 2) through 4) in a control subject not administered the one or more compounds; and 5) comparing the molecular flux rates in the one or more metabolic pathways of interest in the subject administered the one or more compounds to the molecular flux rates in the one or more metabolic pathways in the subject not administered said one or more compounds.

In a further embodiment, the effect on the one or more biomarkers of toxicity of epithelial tissue is chosen from breast epithelial cell proliferation, colon epithelial cell proliferation, prostate epithelial cell proliferation, ovarian epithelial cell proliferation, endometrial cell proliferation, bronchial epithelial cell proliferation, pancreatic epithelial cell proliferation, keratin synthesis in skin, and keratinocyte proliferation.

In yet another embodiment of the invention, the one or more metabolic pathways of interest related to the effect on the one or more biomarkers of toxicity of epithelial tissue are measured in response to a specific dose or a range of doses of the one or more compounds.

In still another embodiment, the collection of the sample is by non-invasive or minimally invasive means for example, by the collection of ductal fluid by lavage, skin scrapings, skin flakings, seminal fluid, nasal secretions, stool (feces), tears, sweat, flatulence, and the like, and epithelial cells and/or their biochemical components are isolated from the collected samples and further analyzed as described supra and infra.

In another embodiment, the molecular flux rates of the one or more metabolic pathways of interest are measured concurrently.

In a further embodiment, the concurrent measurement of the molecular flux rates from said metabolic pathways of interest is achieved by use of stable isotopic labeling techniques and the isotope label used is a stable (i.e., non-radioactive) isotope. In one aspect, the stable isotope used in the stable isotopic labeling is stable isotope-labeled water. In a particular aspect, the stable isotope-labeled water is ²H₂O.

In another embodiment, the concurrent measurement of the molecular flux rates from said metabolic pathways of interest is achieved by use of radioisotope labeling techniques.

In one aspect of the present invention, the one or more compounds is an already-approved drug. In a further aspect, the already-approved drug is a Federal Food and Drug Administration-approved drug.

In one aspect, the already-approved drug is selected randomly. In another aspect, the already-approved drug is selected on the basis of a specific biochemical rationale or hypothesis concerning a hypothesized role in the molecular pathogenesis of one or more diseases of epithelial tissue origin.

In yet another embodiment of the present invention, the one or more compounds is a new chemical entity. In another aspect, the one or more compounds is a biological factor.

In yet another aspect of the present invention, the one or more metabolic pathways of interest are measured in response to a specific dose or a range of doses of the one or more compounds.

In still another aspect of the invention, the one or more metabolic pathways of interest are chosen from breast epithelial cell proliferation, colon epithelial cell proliferation, prostate epithelial cell proliferation, ovarian epithelial cell proliferation, endometrial cell proliferation, bronchial epithelial cell proliferation, pancreatic epithelial cell proliferation, keratin synthesis in skin, and keratinocyte proliferation.

In a further aspect of the present invention, the already-approved drug is screened for actions on multiple biochemical processes concurrently.

In one embodiment of the present invention, the subject is chosen from rabbits, dogs, mice, rats, guinea pigs, pigs, non-human primates, and humans. In a further aspect of the invention, the subject is a human.

In another embodiment of the invention, the isotope labeled substrate is chosen from ²H₂O, ²H-glucose, ²H-labeled amino acids, ²H-labeled organic molecules, ¹³C-labeled organic molecules, ¹³CO₂, ¹⁵N-labeled organic molecules, ³H₂O, ³H-labeled glucose, ³H-labeled amino acids, ³H-labeled organic molecules, ¹⁴C-labeled organic molecules, and ¹⁴CO₂. In a further embodiment, the isotope labeled substrate is ²H₂O.

In yet another embodiment of the invention, the one or more compounds are administered according to established or hypothesized dose ranges that have the potential for biological activity in said subject.

In still another embodiment of the invention, the one or more samples obtained from one or more spaces external to the subject are collected at known times or intervals after administration or contacting said subject to said isotope-labeled substrate and after exposing said subject to said one or more compound.

In yet a further embodiment of the invention, the one or more spaces external to the subject is chosen from the urethra of the penis, vagina, uterus, gastrointestinal tract, respiratory tract, buccal cavity, skin surface, and breast duct.

In another aspect, the one or more samples collected from the one or more spaces external to the subject is chosen from urine, semen, vaginal secretions, stool, gastrointestinal secretion, sputum, skin flakes, and breast fluid.

In another embodiment, combinations of two or more compounds are exposed to the subject.

In yet another embodiment of the invention, synergistic, complementary, or antagonistic actions of combinations of compounds on molecular flux rates through the one or more metabolic pathways are determined by comparing said molecular flux rates in said subject exposed to the combination of compounds to said molecular flux rates in said subject exposed to a single compound alone or not exposed to any of said compounds being tested.

In another embodiment of the invention, isotopically perturbed molecules are provided, said isotopically perturbed molecules comprising one or more stable isotopes. The isotopically perturbed molecules are products of the labeling methods described herein. The isotopically perturbed molecules are collected by sampling techniques known in the art and are analyzed using appropriate analytical tools.

In yet another embodiment of the invention, the isotopically perturbed molecules are labeled with one or more radioactive isotopes.

In yet another embodiment of the invention, one or more kits are provided that comprise isotope-labeled precursors and instructions for using them. The kits may contain stable-isotope labeled precursors or radioactive-labeled isotope precursors or both. Stable-isotope labeled precursors and radioactive-labeled isotope precursors may be provided in one kit or they may be separated and provided in two or more kits. The kits may further comprise one or more tools for administering the isotope-labeled precursors. The kits may also comprise one or more tools for collecting samples from a subject.

In yet another embodiment of the invention, one or more information storage devices are provided that comprise data generated from the methods of the present invention. The data may be analyzed, partially analyzed, or unanalyzed. The data may be imprinted onto paper, plastic, magnetic, optical, or other medium for storage and display.

In yet another embodiment of the invention, one or more drug agents identified and at least partially characterized by the methods of the present invention are contemplated.

In yet another embodiment, the present invention is directed to a method for detecting the presence or absence of an epithelial disease or monitoring an epithelial disease of a subject. The method may include steps a) to e). In step a), an isotope-labeled substrate is administered to a subject for a period of time sufficient for the isotope-labeled substrate to label at least one targeted molecule of interest in one or more metabolic pathways of interest in one or more cells of epithelial origin in the subject. In the method, the subject has one or more body spaces; the body spaces are in communication with the external environment of the subject. In step b), one or more samples are obtained from the one or more body spaces in the subject. The samples include at least one isotope-labeled targeted molecule of interest. In step c), the content, rate of incorporation and/or pattern or rate of change in content and/or pattern of isotope labeling is measured for at least one targeted molecule of interest. In step d), the molecular flux rates are calculated in one or more metabolic pathways of interest based on the content and/or pattern or rate of change of content and/or pattern of isotopic labeling in the at least one targeted molecule of interest. Finally, in step e) the molecular flux rates are measured in the one or more metabolic pathways of interest to detect the presence or absence of the epithelial disease or to monitor the epithelial disease in the subject.

In yet another embodiment, the present invention is directed to a method for detecting the presence or absence of cancerous or pre-cancerous breast cancer or monitoring a treatment of breast cancer in a patient. Pre-cancerous breast cancer is breast cancer in the pre-cancerous state which is generally undetectable by breast exam or mammogram. The method may include steps a) to e). In step a), deuterated water is administered to a patient for a period of time sufficient for the deuterated water to label DNA in one or more breast epithelial cells in the patient. In step b), a sample of the breast epithelial cells including the labeled DNA is collected. In step c), the content, rate of incorporation and/or pattern or rate of change in content and/or pattern of isotope labeling of the DNA in the breast epithelial cells is measured. Finally, in step d), the rate or pattern to historical data, data obtained from a control subject, data obtained from non-cancerous tissue in the same subject, data obtained from the same subject prior to treatment, data obtained from the same subject prior to cancer onset is compared in order to evaluate whether or not there is an increase or decrease in breast epithelial cell proliferation with respect to normal subjects or tissues, in order to detect the presence or absence of cancerous or pre-cancerous breast cancer, or monitor a treatment of breast cancer in the patient.

In yet another embodiment, the present invention is directed to a method for detecting the presence or absence of prostate cancer or monitoring a treatment of prostate cancer in a patient. In detecting the presence of prostate cancer, the method allows detection of pre-cancerous conditions often undectable by general techniques such as prostate examination and prostate specific antigen assays. The method can include steps a) to e). In step a) deuterated water is administered to a patient for a period of time sufficient for the deuterated water to label DNA in one or more prostate epithelial cells in the patient. In step b), a sample of the prostate epithelial cells including the labeled DNA is collected. In step c) the content, rate of incorporation and/or pattern or rate of change in content and/or pattern of isotope labeling of said DNA in said prostate epithelial cells is compared. In step d), the rate or pattern to historical data, data obtained from a control subject, data obtained from tissue in the same subject, data obtained from the same subject prior to treatment, or data obtained from the same subject prior to cancer onset is compared in order to evaluate whether or not there is an increase or decrease in prostate epithelial cell proliferation with respect to normal subjects or tissues, to detect the presence or absence of prostate cancer, or monitor treatment of prostate cancer in the patient.

In yet another embodiment, the present invention is directed to a method for detecting the presence or absence of a skin disorder or monitoring a treatment of a skin disorder in a patient. In the method, the skin disorder can be detected early in the development of the disorder. The method may include steps a) through e). In step a) deuterated water is administered to a patient for a period of time sufficient for the deuterated water to label keratin in the skin of the patient. In step b), a sample of the labeled keratin is collected. In step c), the content, rate of incorporation and/or pattern or rate of change in content and/or pattern of isotope labeling of the keratin is determined. In step d), the rate or pattern to historical data, data obtained from a control subject, data obtained from non-disordered tissue in the same subject, data obtained from the same subject prior to treatment, or data obtained from the same subject prior to disorder onset is compared in order to evaluate whether or not there is an increase or decrease in keratin synthesis with respect to normal subjects or tissues, to detect the presence or absence of a skin disorder or to monitor the treatment of a skin disorder.

The skin disorder may be any skin disorder as disclosed herein. In one format, the skin disorder is psoriasis. In another format, the skin disorder is eczema.

In yet another embodiment, the present invention is directed to a method method for detecting the presence or absence of a colon disorder or monitoring a treatment of a colon disorder in a patient. The method may include steps a)-d). In step a), deuterated water is administered to the patient for a period of time sufficient for the deuterated water to label mucin in one or more colon epithelial cells in the patient. In step b), a sample of stool or epithelial tissue or colon epithelial cells including the labeled mucin is collected. In step d), the content, rate of incorporation and/or pattern or rate of change in content and/or pattern of isotope labeling of the mucin in the colon epithelial cells is measured. In step d), the rate or pattern to historical data, data obtained from a control subject, data obtained from non-disorder tissue in the same subject, data obtained from the same subject prior to treatment, or data obtained from the same subject prior to disorder onset is compared in order to evaluate whether or not there is an increase or decrease in mucin synthesis with respect to normal subjects or tissues, to detect the presence or absence of a colon disorder or monitor the treatment of a colon disorder.

The colon disorder may be any colon disorder as disclosed herein. In one format, the colon disorder is colon cancer, irritable bowl disease or Crohn's disease.

In yet another embodiment, the present invention is directed to a method for detecting the presence or absence of a colon disorder or monitoring a treatment of a colon disorder in a patient. In detecting the presence of a colon disorder, the method allows detection of pre-cancerous or other early-sign indications often undectable by traditional colon analysis techniques such as colon examination including colonoscopies. The method may include steps a) to e). In step a), deuterated water is administered to a patient for a period of time sufficient for the deuterated water to label DNA in one or more colon epithelial cells in the patient thereby forming labeled colon epithelial cell DNA. In step b), a sample of stool or tissue including the labeled colon epithelial cell DNA is collected. In step c) the content, rate of incorporation and/or pattern or rate of change in content and/or pattern of isotope labeling of said DNA in the colon epithelial cells is measured. In step d) the rate or pattern to historical data, data obtained from a control subject, data obtained from non-disorder tissue in the same subject, data obtained from the same subject prior to treatment, or data obtained from the same subject prior to disorder onset is compared in order to evaluate whether or not there is an increase or decrease in colon epithelial cell proliferation with respect to normal subjects or tissues, to detect the presence or absence of a colon disorder or monitor the treatment of a colon disorder.

### BRIEF DESCRIPTION OF THE DRAWINGS

FIG. 1 is a schematic diagram showing one embodiment of the invention whereby labeled epithelial cells or molecules are collected, isolated, and analyzed to obtain useful information in assessing such things as cancer risk or chemotherapeutic efficacy.

FIG. 2 shows the proliferation of breast epithelial cells (BEC's) as described in Example 1. FIG. 2A depicts flow cytometry results of BEC's isolated from the ductal lavage (DL) fluid of women, showing >95% purity. Cells stained with anti-cytokeratin 18 antibody conjugated to FITC compared to control irrelevant-FITC antibody. >95% of cells are positive for cytokeratin 18, an intracellular marker for luminal BEC. FIG. 2B depicts the calculated proliferation rates of BEC isolated from DL fluid, after ²H₂O label administration in vivo for 14 days. Proliferation of BEC collected by DL from two healthy women. Subjects were labeled with ²H₂O for 14 days prior to DL. FIG. 2C depicts the proliferation of BEC's isolated from biopsy specimens from normal and tumourous breast tissue. This figure illustrates the types of proliferative differences observed in epithelial cells in the cancerous state, and also shows that the observed rate of proliferation of BEC's in healthy tissue is similar when measured by the two different methods.

FIG. 3 depicts prostate epithelial cell (PEC) proliferation. FIG. 3A depicts flow cytometric isolation of prostate epithelial cells (PEC's) isolated from seminal fluid, demonstrating >95% purity. The left panel depicts total cells, the central panel depicts the cells removed, and the right panel depicts the >95% pure PEC's isolated by cell sorting. FIG. 3B depicts the calculated rates of PEC proliferation in five male subjects as determined using the techniques described in example 2, infra. Subjects 3 and 5 had accelerated PEC proliferation. Subject 5 was later determined to have benign prostate hyperplasia, evidenced by the increased proliferation observed in seminal PEC's. No additional clinical data could be obtained for subject 3. Subjects 1,2, and 4 were normal subjects.

FIG. 4 depicts deuterium enrichment in keratin from skin strips obtained from normal mice (left) and psoriatic FSN mice (right). In the psoriasis disease model (the FNS mice) skin proliferates approximately 3 times more rapidly than in control animals.

FIG. 5 depicts deuterium enrichment in keratin from skin strips obtained from a healthy adult human. Deuterium incorporation reaches its maximum at about 5 weeks, the period of time it takes for the skin to completely replace itself.

FIG. 6 is a schematic diagram showing the drug discovery, development, and approval (DDA) process using effects on epithelial biomarkers (i.e., data collected by the methods of the present invention) as a means for deciding to continue or cease efforts.

FIG. 7 illustrates use of the methods and compositions disclosed herein in a drug discovery process.

### DETAILED DESCRIPTION OF THE INVENTION

I. Introduction

Epithelial cancers are the most common and medically important oncologic disorders in the Western world. Neoplasias of epithelial tissues include cancers of the breast, colon, prostate, lung (bronchus), pancreas, hepatobiliary system, liver (hepatoma), head and neck (nasopharyngeal), kidney (hypernephroma), uterus (endometrium), vagina, uterine cervix, ovary, bladder, skin (basal cell and malignant melanoma), urogenital system, urethra, conjunctiva, and others. Epithelial cancers derive from epithelial tissues which are tissues that communicate topologically with the space outside of the body of a metazoan organism. This communication may take the form of a luminal surface (i.e., a tubular structure with epithelial cell lining) or an integumental surface (e.g., skin, mucosa of the lip or tongue, etc.). The cells of an epithelial tissue are therefore either directly or indirectly exposed to and in contact with the outside environment. This feature has allowed development of certain clinically non-invasive (or minimally invasive) diagnostic procedures for disorders involving selected epithelial tissues. Examples of such procedures include cervical swabs (pap smears), fecal occult blood testing (hemoccult), urine cytology, sputum cytology, and breast nipple aspirate cytology for cancer screening or diagnosis.

The carcinogenic process in epithelial tissues, as in other tissues, is characterized by an extremely long pre-cancer phase (1,2). Indeed, carcinogenesis is believed to be a multi-step process that evolves over decades, starting with a genetic damage event (initiation) and proceeding through a series of events (promotional phase) that culminates in a clinically apparent neoplastic growth. The proliferation of initiated cells—i.e., the process of mitotic cell division, involving DNA replication—is a critical component of the promotional phase of epithelial carcinogenesis (1-3). It has often been stated (3) that the two driving forces of carcinogenesis are “mutagenesis and mitogenesis.”

For this reason, many strategies to prevent epithelial cancers that have been proposed and tested are based on the goal of reducing proliferation of cells in the target epithelial tissue (i.e., reducing mitogenesis). The target cells here are initiated but normal (i.e., pre-cancer) cells in a tissue. Examples include anti-estrogens (e.g., tamoxifen, raloxifen) to reduce mammary epithelial cell proliferation and prevent the development of breast cancer; dietary components (e.g., calcium, fiber, low-fat) or cyclo-oxygenase (COX) inhibitors (e.g., COX-2 inhibitors, salicylates) to reduce colon epithelial cell (colonocyte) proliferation and prevent colon cancer; anti-androgens (e.g., finasteride) to reduce prostate epithelial cell proliferation and prevent prostate cancer; and others. These strategies have met with some success and represent a highly promising area of future medical practice.

At present, however, there are few clinical diagnostic methods available for reliably monitoring either the natural evolution of epithelial carcinogenesis or for evaluating the efficacy of interventions, such as anti-proliferative agents, aimed at reducing cancer risk. As noted, supra, the topologic communication of epithelial tissues with the outside space has allowed for some useful cytologic or other techniques known in the art for identifying relatively late-stage cancers. The most successful of these are the pap smear for cervical cancer, stool hemoccult testing for colon cancer, and sputum cytology for lung cancer. With the exception of pap smears, however, these diagnostic modalities require phenotypically apparent, advanced neoplastic changes, or pathologic tissue events (such as bleeding) and are therefore not useful as biomarkers in the long pre-cancer phase. Because the pre-cancer phase is in principle the optimal period for cancer prevention (1-3), these tests are not of value for preventative efforts. The absence of biomarkers of cancer chemoprevention efficacy has been noted by numerous authorities (1,2) to be one of the leading problems and highest priorities in the field of cancer prevention.

Epithelial tissues are also affected by numerous important non-cancerous disorders that involve altered proliferation or turnover of epithelial cells. These include benign prostatic hyperplasia (BPH) and prostatitis; colitis and colon polyps; cystic disorders of the breast, ovaries, kidney, and liver; bronchitis, sinusitis, pharyngitis, and other acute or chronic infections of the upper respiratory system; primary biliary cirrhosis; endometriosis and endometritis; vaginitis and vaginosis; cervicitis and cervical infections; bladder infections, bladder wall hyperplasia and other bladder disturbances; dermatitis, eczema, psoriasis, and other hyperproliferative skin conditions; benign tumors or masses of the liver, endometrium, and breast; and many others known in the art.

Some of the non-cancerous epithelial conditions listed, supra, rank among the most common, troubling, and disabling disorders of modern humans—e.g., psoriasis, BPH, ulcerative colitis, chronic bronchitis, and bladder wall disturbances among others. The capability of monitoring the activity of these disorders in a non-invasive (or minimally invasive) manner, by virtue of their topological communication with the outside world, would have potentially enormous clinical utility, but no such tests are currently available. In the field of psoriasis, for example, current therapeutics are not optimally effective and this is in large part because the objective measures of psoriatic disease activity are lacking (4). Without such markers, evaluation of treatment efficacy has been compromised (i.e., metrics are highly subjective, have poor inter-observer reproducibility, are susceptible to placebo effects, etc.) in clinical studies and in routine medical practice.

The availability of a reliable biomarker underlying psoriatic activity, as distinguished from symptoms or clinical signs, would therefore represent a major advance in this common condition (4). The instant invention describes such a biomarker for psoriasis. Psoriasis is characterized by hyperproliferation of keratinocytes (epidermal epithelial cells) and rapid turnover of their primary biochemical product (keratin); the disclosed methods allow one to measure the hyperproliferation of keratinocytes by using de novo DNA synthesis and keratin turnover (keratin synthesis and degradation) as two biomarkers of psoriasis (see discussion of methods, infra).

Disclosed herein are methods that provide a generally applicable non-invasive approach for evaluating risk of cancer or other diseases of epithelial tissues and for monitoring therapeutic interventions in epithelial tissues. The methods are non-invasive or minimally invasive and easily repeatable in an individual over time. The disclosed methods are applicable to many, if not most, of the epithelial tissues that become important cancers and provide key information early in the carcinogenic process (i.e., before crude phenotypic or tissue pathologic changes have developed). The methods generate particularly useful information such as cell proliferation and death rates (cell turnover kinetics), DNA repair processes, protein turnover, and other biochemical events that characterize the pre-cancer phase of epithelial carcinogenesis and also events that characterize disease progression of non-cancerous epithelial diseases and conditions.

The instant invention discloses methods that embody the discovery that (1) epithelial cells (often intact) or important biochemical components (molecules) from epithelial cells can be isolated from excreta; (2) the isotopic content or pattern of these cells or molecules isolated from excreta can be measured using analytic procedures that have been developed by the Applicant or that are known in the art, after administration in vivo to the individual of targeted, isotopically perturbed metabolic precursors, prior to collection of excreta; and (3) the isotopic content or pattern of these cells or molecules isolated from excreta can reveal key information about cell proliferation or other biochemical events (e.g., protein turnover) that characterize the early phase of epithelial carcinogenesis or that are involved in other disorders of epithelial tissues.

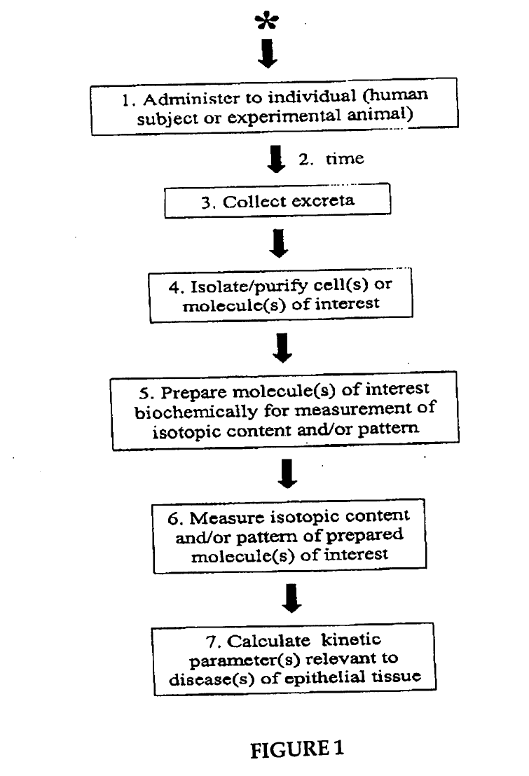

By combining the discoveries, supra, into a single procedure (see FIG. 1), the Applicant herein has disclosed a generally applicable, non-invasive (or minimally invasive) method for generating information about diseases of epithelial tissue, including cancer. This combined method thereby fulfills the need for non-invasive or minimally invasive biomarkers of disease risk, activity, progression, pathogenesis, mechanism, or therapeutic response for diseases of epithelial tissues, including epithelial cancers.

The present invention combines the following elements in a manner that solves the need for non-invasive biomarkers of epithelial diseases such as, inter alia, cancer, psoriasis, ulcerative colitis, primary biliary cirrhosis, benign prostatic hyperplasia, chronic bronchitis, bladder hyperplasia, etc.:
Epithelial tissues communicate with the space outside of the body, so that their excreta (luminal or integumental) is often accessible to collection or sampling, by non-invasive or minimally invasive techniques
Excreta from epithelial tissues (luminal or integumental) often contains epithelial cells or molecules released from epithelial cells
In vivo labeling of epithelial cell biochemical processes (e.g., as DNA replication and cell proliferation) can reveal important and even critical information relevant to the initiation, progression, activity, severity, regression, pathogenesis, mechanism, or therapeutic response of a disease of the epithelial tissue of interest, that is ascertainable by measurements of the isotopic content and/or pattern of epithelial cells or molecules of interest from epithelial cells.
   II. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999). Furthermore, procedures employing commercially available assay kits and reagents will typically be used according to manufacturer-defined protocols unless otherwise noted.

III. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999). As appropriate, procedures involving the use of commercially available kits and reagents are generally carried out in accordance with manufacturer defined protocols and/or parameters unless otherwise noted.

“Molecular flux rates” refers to the rate of synthesis and/or breakdown of molecules within a cell, tissue, or organism. “Molecular flux rates” also refers to a molecule's input into or removal from a pool of molecules, and is therefore synonymous with the flow into and out of said pool of molecules.

“Metabolic pathway” refers to any linked series of two or more biochemical steps in a living system (i.e., a biochemical process), the net result of which is a chemical, spatial or physical transformation of a molecule or molecules. Metabolic pathways are defined by the direction and flow of molecules through the biochemical steps that comprise the pathway. Molecules within metabolic pathways can be of any biochemical class, e.g., including but not limited to lipids, proteins, amino acids, carbohydrates, nucleic acids, polynucleotides, porphyrins, glycosaminoglycans, glycolipids, intermediary metabolites, inorganic minerals, ions, etc.

“Flux rate through a metabolic pathway” refers to the rate of molecular transformations through a defined metabolic pathway. The unit of flux rates through pathways is chemical mass per time (e.g., moles per minute, grams per hour). Flux rate through a pathway optimally refers to the transformation rate from a clearly defined biochemical starting point to a clearly defined biochemical end-point, including all the stages in between in the defined metabolic pathway of interest.

“Isotopes” refer to atoms with the same number of protons and hence of the same element but with different numbers of neutrons (e.g., ¹H vs. ²H or D).

“Isotopologues” refer to isotopic homologues or molecular species that have identical elemental and chemical compositions but differ in isotopic content (e.g., CH₃NH₂ vs. CH₃NHD in the example above). Isotopologues are defined by their isotopic composition, therefore each isotopologue has a unique exact mass but may not have a unique structure. An isotopologue is usually comprised of a family of isotopic isomers (isotopomers) which differ by the location of the isotopes on the molecule (e.g., CH₃NHD and CH₂DNH₂ are the same isotopologue but are different isotopomers).

“Isotope-labeled water” includes water labeled with one or more specific heavy isotopes of either hydrogen or oxygen. Specific examples of isotope-labeled water include ²H₂O, ³H₂O, and H₂¹⁸O.

“Chemical entity” includes any molecule, chemical, or compound, whether new or known, that is administered to a living system for the purpose of screening it for biological or biochemical activity toward the goal of discovering potential therapeutic agents (drugs or drug candidates or drug leads) or uncovering toxic effects (industrial chemicals, pesticides, herbicides, food additives, cosmetics, and the like).

“Drug leads” or “drug candidates” are herein defined as chemical entities or biological molecules that are being evaluated as potential therapeutic agents (drugs). “Drug agents” or “agents or “compounds” are used interchangeably herein and describe any composition of matter (e.g., chemical entity or biological factor) that is administered, approved or under testing as potential therapeutic agent or is a known therapeutic agent.

“Known drugs” or “known drug agents” or “already-approved drugs” refers to agents (i.e., chemical entities or biological factors) that have been approved for therapeutic use as drugs in human beings or animals in the United States or other jurisdictions. In the context of the present invention, the term “already-approved drug” means a drug having approval for an indication distinct from an indication being tested for by use of the methods disclosed herein. Using psoriasis and fluoxetine as an example, the methods of the present invention allow one to test fluoxetine, a drug approved by the FDA (and other jurisdictions) for the treatment of depression, for effects on biomarkers of psoriasis (e.g., keratinocyte proliferation or keratin synthesis); treating psoriasis with fluoxetine is an indication not approved by FDA or other jurisdictions. In this manner, one can find new uses (in this example, anti-psoriatic effects) for an already-approved drug (in this example, fluoxetine).

“Biological factor” refers to a compound or compounds made by living organisms having biological or physiological activities (e.g., preventive, therapeutic and/or toxic effects). Examples of biological factors include, but are not limited to, vaccines, polyclonal or monoclonal antibodies, recombinant proteins, isolated proteins, soluble receptors, gene therapy products, and the like. As used herein, the term “biologics” is synonymous with “biological factor.”

“Compound” means, in the context of the present invention, any new chemical entity, chemical entity, drug lead, drug candidate, drug, drug agent, agent, known drug, known drug agent, already-approved drug, biologic, or biological factor. The term is meant to encompass all chemical and biological molecules.

“Food additive” includes, but is not limited to, organoleptic agents (i.e., those agents conferring flavor, texture, aroma, and color), preservatives such as nitrosamines, nitrosamides, N-nitroso substances and the like, congealants, emulsifiers, dispersants, fumigants, humectants, oxidizing and reducing agents, propellants, sequestrants, solvents, surface-acting agents, surface-finishing agents, synergists, pesticides, chlorinated organic compounds, any chemical ingested by a food animal or taken up by a food plant, and any chemical leaching into (or otherwise finding its way into) food or drink from packaging material. The term is meant to encompass those chemicals which are added into food or drink products at some step in the manufacturing and packaging process, or find their way into food by ingestion by food animals or uptake by food plants, or through microbial byproducts such as endotoxins and exotoxins (pre-formed toxins such as botulinin toxin or aflatoxin), or through the cooking process (such as heterocyclic amines, e.g., 2-amino-3-methyllimidazo[4,5-f]quinolone), or by leaching or some other process from packaging material during manufacturing, packaging, storage, and handling activities.

“Industrial chemical” includes, but is not limited to, volatile organic compounds, semi-volatile organic compounds, cleaners, solvents, thinners, mixers, metallic compounds, metals, organometals, metalloids, substituted and non-substituted aliphatic and acyclic hydrocarbons such as hexane, substituted and non-substituted aromatic hydrocarbons such as benzene and styrene, halogenated hydrocarbons such as vinyl chloride, aminoderivatives and nitroderivatives such as nitrobenzene, glycols and derivatives such as propylene glycol, ketones such as cyclohexanone, aldehydes such as furfural, amides and anhydrides such as acrylamide, phenols, cyanides and nitrites, isocyanates, and pesticides, herbicides, rodenticides, and fungicides.

“Environmental pollutant” includes any chemical not found in nature or chemicals that are found in nature but artificially concentrated to levels exceeding those found in nature (at least found in accessible media in nature). So, for example, environmental pollutants can include any of the non-natural chemicals identified as an occupational or industrial chemical yet found in a non-occupational or industrial setting such as a park, school, or playground. Alternatively, environmental pollutants may comprise naturally occurring chemicals such as lead but at levels exceeding background (for example, lead found in the soil along highways deposited by the exhaust from the burning of leaded gasoline in automobiles). Environmental pollutants may be from a point source such as a factory smokestack or industrial liquid discharge into surface or groundwater, or from a non-point source such as the exhaust from cars traveling along a highway, the diesel exhaust (and all that it contains) from buses traveling along city streets, or pesticides deposited in soil from airborne dust originating in farmlands. As used herein, “environmental contaminant” is synonymous with “environmental pollutant.”

“Living system” includes, but is not limited to, cells, cell lines, animal models of disease, guinea pigs, rabbits, dogs, cats, other pet animals, mice, rats, non-human primates, and humans.

“Excreta” is defined herein as any liquid, solid, or gaseous material that is released from the body to the outside world. Examples include, but are not limited to, stool (feces), urine, seminal fluid, sputum, breast ductal fluid, saliva, cervical secretions, vaginal secretions, skin scrapings, skin flakes, hair, nasal secretions, pancreatic-biliary secretions, lacrimal fluid (tears), sweat, flatus, exhaled respiratory gases, or other physical materials released from the body.

A “biological sample” encompasses any sample obtained from a cell, tissue, or organism if such sample was derived from an external space, that is, a luminal or integumentary space external to the body of the organism. The sample may be solid in nature. The definition also encompasses liquid samples of biological origin, that are accessible from an organism through sampling by minimally invasive or non-invasive approaches (e.g., urine collection, needle aspiration, breast fluid collection from breast ductal lavage, skin scraping, semen collection, vaginal secretion collection, nasal secretion collection, sputum collection, stool collection, and other procedures involving minimal risk, discomfort or effort). The definition also includes samples that have been manipulated in any way after their procurement, such as by treatment with reagents, solubilization, or enrichment for certain components, such as proteins, lipids, carbohydrates, or organic metabolites. The term “biological sample” also encompasses a clinical sample such as biological fluid or tissue sample.

By “body spaces in communication with the external environment” is meant any body space having contact with any media of the external environment. This contact may not be direct, but may in fact be indirect with contact by way of a duct or other body cavity ultimately having direct contact with any media of the external environment. For example, intestinal epithelial cells sloughed off from the gastrointestinal (GI) epithelial membrane are in a body space in communication with the external environment when they are located within, or associated with, the lumen of the gastrointestinal tract. Similarly, any intestinal epithelial molecules located within the lumen of the GI tract are also located within a body space in communication with the external environment. Skin cells (keratinocytes) residing on or associated with the surface of the epidermis are also in a body space in communication with the external environment. Furthermore, breast epithelial cells contained within breast fluid within a breast duct are in a body space in communication with the external environment as are prostate epithelial cells contained within seminal fluid found within the ductal system of the male reproductive and sex organs. Further examples include, but are not limited to: cervical epithelial cells within the vagina; vaginal epithelial cells within the vagina; colon epithelial cells (colonocytes) within or associated with stool; endometrial cells within the uterus; bladder epithelial cells located within or associated with the lumen of the bladder or are contained within urine collected outside the body; and bronchial epithelial cells located within, or associated with, the lumen of the airways (e.g., bronchi, bronchioli, alveolar sacs).

“Biological fluid” refers to, but is not limited to, urine, edema fluid, saliva, lacrimal fluid, inflammatory exudates, synovial fluid, abscess, empyema or other infected fluid, sweat, pulmonary secretions (sputum), seminal fluid, feces, bile, intestinal secretions, vaginal secretions, or any other biological fluid found in spaces external to the body (i.e., luminal or integumentary spaces).

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Mass isotopomer” refers to family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nominal mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as M₀; for most organic molecules, this is the species containing all ¹²C, ¹H, ¹⁶O, ¹⁴N, etc. Other mass isotopomers are distinguished by their mass differences from M₀ (M₁, M₂, etc.). For a given mass isotopomer, the location or position of isotopes within the molecule is not specified and may vary (i.e., “positional isotopomers” are not distinguished).

“Mass isotopomer envelope” refers to the set of mass isotopomers comprising the family associated with each molecule or ion fragment monitored.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonomously with the term “mass isotopomer pattern.”

“Monoisotopic mass” refers to the exact mass of the molecular species that contains all ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m₀ and the masses of other mass isotopomers are identified by their mass differences from m₀ (m₁, m₂, etc.).

“Isotopically perturbed” refers to the state of an element or molecule that results from the explicit incorporation of an element or molecule with a distribution of isotopes that differs from the distribution that is most commonly found in nature, whether a naturally less abundant isotope is present in excess (enriched) or in deficit (depleted).

“Metabolic precursors” or “precursors” refer to molecules or atoms that enter into molecular end-products of interest through the metabolic processes of the cell or organism (i.e., through biosynthetic, degradative, and/or intermediary metabolic pathways).

“Cells and molecules” refer to intact (though not necessarily viable or living) cells and to biochemical entities present in the cells.

In the context of the present invention, a “cell of epithelial origin” refers to an epithelial cell or any cell derived from epithelial tissue whether viable or not.

By “molecule of interest” is meant any molecule (polymer and/or monomer), including but not limited to, amino acids, carbohydrates, fatty acids, peptides, sugars, lipids, nucleic acids, polynucleotides, glycosaminoglycans, polypeptides, or proteins that are present within a metabolic pathway within a cell. In the context of the present invention, a “molecule of interest” may be a “biomarker” of an epithelial disease and its flux rate, relative to the flux rate of an unexposed or otherwise healthy subject (i.e., control subject), may represent clinically non-observant or subtle pathophysiological occurrences in a subject of interest that may be predictive of future disease or injury in the subject. In this manner, comparing the flux rates of one or more biomarkers of interest in a subject with the flux rates of one or more biomarkers of interest in a control subject, will find use in diagnosing the subject with, or evaluating or quantifying the subject's risk in acquiring, an epithelial disease of interest. Moreover, such information will find use in establishing a prognosis for a subject having an epithelial disease of interest, monitoring the progression of an epithelial disease of interest in a subject, or evaluating the therapeutic efficacy of a treatment regimen in a subject having an epithelial disease of interest. Molecules of interest may include DNA from epithelial cells that are correlated with, associated with or causative of an epithelial disease (e.g., DNA from prostate epithelial cells, which are involved in prostate cancer or benign prostatic hyperplasia, or from breast epithelial cells, which are involved in breast cancer). Molecules of interest may include proteins from epithelial cells or from secretions of epithelial cells or tissues, or fluids or samples of material related to epithelial cells (e.g., keratin from skin). Molecules of interest may also include other biomolecules including proteoglycans, glycoproteins, carbohydrates, complex carbohydrates, lipids, lipoproteins, mucopolysaccharides, co-factors, signaling molecules, peptides, hormones, steroid hormones, amino acids, or modified amino acids that are secreted by, contained within, associated with, in contact with, or derived from samples of epithelial cells. (e.g, mucin from the intestinal lining). Furthermore, for the purposes of the present invention, molecules of interest are sometimes isolated from excreta, such as stool, that are biologically active (i.e., metabolic processes occur within the excreta, an example being the metabolic processing of excreta by bacteria in stool), molecules of interest may actually be the metabolic products of these biological activities, which may convert the initial labeled molecule of interest into a different, but still labeled, biomolecule. By way of example, genomic DNA derived from colon epithelial cells is converted to oligonucleotides by bacterial nucleases derived from bacteria residing in the colon.

By “subject” is meant a human or animal having an epithelial disease or condition or having some level of risk in acquiring an epithelial disease or condition.

By “control subject” is meant a human or animal not having the epithelial disease or condition or not having some level of risk in acquiring the epithelial disease or condition.

“Monomer” refers to a chemical unit that combines during the synthesis of a polymer and which is present two or more times in the polymer. “Polymer” refers to a molecule synthesized from and containing two or more repeats of a monomer. A “biopolymer” is a polymer synthesized by or in a living system or otherwise associated with a living system.

“Protein” refers to a polymer of amino acids. As used herein, a “protein” may refer to long amino acid polymers as well as short polymers such as peptides.

By “amino acid” is meant any amphoteric organic acid containing the amino group (i.e., NH₂). The term encompasses the twenty common (often referred in the art as “standard” or sometimes as “naturally occurring”) amino acids as well as the less common (often referred in the art as “nonstandard”) amino acids. Examples of the twenty common amino acids include the alpha-amino acids (or α-amino acids), which have the amino group in the alpha position, and generally have the formula RCH—(NH₂)—COOH. The α-amino acids are the monomeric building blocks of proteins and can be obtained from proteins through hydrolysis. Examples of nonstandard amino acids include, but are not limited to γ-aminobutyric acid, dopamine, histamine, thyroxine, citrulline, ornithine, homocysteine, and S-adenosylmethionine.

“Lipid” refers to any of a heterogeneous group of fats and fatlike substances characterized by being water insoluble and being extractable by nonpolar (or organic) solvents such as alcohol, ether, chloroform, benzene, etc. All contain as a major constituent aliphatic hydrocarbons. The lipids, which are easily stored in the body, serve as a source of fuel, are an important constituent of cell structure, and serve other biological functions. Lipids include, but are not limited to fatty acids, neutral fats (e.g., triacylglycerols), waxes and steroids (e.g., cholesterol). Complex lipids comprise the glycolipids, lipoproteins and phospholipids.

“Fatty acids” are carboxylic acids with long-chain hydrocarbon side groups. They are comprised of organic, monobasic acids, which are derived from hydrocarbons by the equivalent of oxidation of a methyl group to an alcohol, aldehyde, and then acid. Fatty acids can be either saturated or unsaturated.

By “DNA” is meant a polymeric form of deoxyribonucleotides (adenine, guanine, thymine, or cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. The term captures molecules that include the four bases adenine, guanine, thymine, or cytosine, as well as molecules that include base analogs which are known in the art.

A “nucleic acid” sequence refers to a DNA or RNA sequence. The term captures sequences that include any of the known base analogues of DNA and RNA such as, but not limited to 4-acetylcytosine, 8-hydroxy-N⁶-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N⁶-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N⁶-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

By “carbohydrate” is meant an aldehyde or ketone derivative of a straight-chain polyhydroxyl alcohol containing at least three carbon atoms. The polyhydroxyl alcohol is primarily (but not exclusively) of the pentahydric and hexahydric alcohol varieties. Carbohydrates are so named because the hydrogen and oxygen are usually in the proportion to form water with the general formula Cₙ(H₂O)ₙ. The most important carbohydrates are the starches, sugars, celluloses and gums. They are classified into mono, di, tri, poly and heterosaccharides. The smallest are monosaccharides like glucose whereas polysaccharides such as starch, cellulose or glycogen can be large and indeterminate in length.

By “sugar” is meant the common name for any sweet, crystalline, simple carbohydrate that is an aldehyde or ketone derivative of a polyhydric alcohol. Sugars are mainly disaccharides like sucrose and monosaccharides like fructose or glucose. The term encompasses monosaccharides, disaccharides, trisaccharides, heterosaccharides, or polysaccharides (which are comprised of monosaccharide residues). Monosaccharides include glucose (both D-glucose and L-glucose), mannose, fructose galactose and sugar derivatives including, but not limited to N-acetylmuramic acid, N-acetyineuraminic acid and other sialic acids, N-acetylmannosamine, glucuronic acid, glucosamine, etc. Polysaccharides include disaccharides such as sucrose, maltose and lactose and longer chain sugar molecules such as starch, glycogen, cellulose, chitin, etc. By the term “oligosaccharide” is meant a molecule comprised of a few covalently linked monosaccharide monomers.

By “glycosaminoglycan” is meant a polymer comprised of a network of long, unbranched chains made up of repeating units of disaccharides that contain amino group sugars, at least one of which has a negatively charged side group (carboxylate or sulfate). Examples of glycosaminoglycans include, but are not limited to hyaluronate (D-glucuronic acid-N-acetyl-D-glucosamine: MW up to 10 million), chondroitin sulfate (D-glucuronic acid-N-acetyl-D-galactosamine-4 or 6-sulfate), dermatan sulfate (D-glucuronic acid or L-iduronic acid-N-acetyl-D-galactosamine), keratan sulfate (D-galactose-N-acetyl-D-glucosamine sulfate), and heparan sulfate (D-glucuronic acid or L-iduronic acid-N-acetyl-D-glucosamine). “Mucopolysaccharide” is a term that is synonymous with glycosaminoglycan.

By “glycoprotein” is meant a protein or polypeptide that is covalently linked to one or more carbohydrate molecules. Glycoproteins include proteoglycans and many, if not most, of the important integral membrane proteins protruding through the exterior leaflet into the extracellular space, as well as many, if not most, of the secreted proteins.

By “proteoglycan” is meant any of a diverse group of macromolecules comprising proteins and glycosaminoglycans. “Mucoprotein” is a term that is synonymous with proteoglycan.

“Isotope labeled substrate” includes any isotope-labeled precursor molecule that is able to be incorporated into a molecule of interest in a living system. Examples of isotope labeled substrates include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃.

“Labeled sugar” refers to a sugar incorporating one or more ²H isotopes.

“Labeled fatty acid” refers to a fatty acid incorporating one or more ²H isotopes.

“Deuterated water” refers to water incorporating one or more ²H isotopes.

“Labeled glucose” refers to glucose labeled with one or more ²H isotopes. Specific examples of labeled glucose or ²H-labeled glucose include [6,6-²H₂]glucose, [1-²H₁]glucose, and [1,2,3,4,5,6-²H₇] glucose.

“Administer[ed]” includes a living system exposed to a chemical entity or entities (including exposure to metabolic precursors). Such exposure can be from, but is not limited to, topical application, oral ingestion, inhalation, subcutaneous injection, intraperitoneal injection, intravenous injection, and intraarterial injection, in animals or other higher organisms.

By “toxic effect” is meant an adverse response by a living system to a chemical entity or known drug agent. A toxic effect can be comprised of, for example, end-organ toxicity.

An “individual” is a vertebrate, preferably a mammal, more preferably a human.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

“At least partially identified” in the context of drug discovery and development means at least one clinically relevant pharmacological characteristic of a drug agent (i.e., a “compound”) has been identified using one or more of the methods of the present invention. This characteristic may be a desirable one, for example, increasing or decreasing molecular flux rates through a metabolic pathway that contributes to a disease process, altering signal transduction pathways or cell surface receptors that alter the activity of metabolic pathways relevant to a disease, inhibiting activation of an enzyme and the like. Alternatively, a pharmacological characteristic of a drug agent may be an undesirable one for example, the production of one or more toxic effects. There are a plethora of desirable and undesirable characteristics of drug agents well known to those skilled in the art and each will be viewed in the context of the particular drug agent being developed and the targeted disease. Of course, a drug agent can be more than at least partially identified when, for example, when several characteristics have been identified (desirable or undesirable or both) that are sufficient to support a particular milestone decision point along the drug development pathway. Such milestones include, but are not limited to, pre-clinical decisions for in vitro to in vivo transition, pre-IND filing go/no go decision, phase I to phase II transition, phase II to phase III transition, NDA filing, and FDA approval for marketing. Therefore, “at least partially” identified includes the identification of one or more pharmacological characteristics useful in evaluating a drug agent in the drug discovery/drug development process. A pharmacologist or physician or other researcher may evaluate all or a portion of the identified desirable and undesirable characteristics of a drug agent to establish its therapeutic index. This may be accomplished using procedures well known in the art.

“Manufacturing a drug agent” in the context of the present invention includes any means, well known to those skilled in the art, employed for the making of a drug agent product. Manufacturing processes include, but are not limited to, medicinal chemical synthesis (i.e., synthetic organic chemistry), combinatorial chemistry, biotechnology methods such as hybridoma monoclonal antibody production, recombinant DNA technology, and other techniques well known to the skilled artisan. Such a product may be a final drug agent that is marketed for therapeutic use, a component of a combination product that is marketed for therapeutic use, or any intermediate product used in the development of the final drug agent product, whether as part of a combination product or a single product. “Manufacturing drug agent” is synonymous with “manufacturing a compound.”

By “biomarker” is meant a biochemical measurement from the organism which is useful or potentially useful for measuring the initiation, progression, severity, pathology, aggressiveness, grade, activity, disability, mortality, morbidity, disease sub-classification or other underlying pathogenic or pathologic feature of one or more epithelial diseases. The concept of a biomarker also includes a physical measurement on the body, such as blood pressure, which is useful for measuring the initiation, progression, severity, pathology, aggressiveness, grade, activity, disability, mortality, morbidity, disease sub-classification or other underlying pathogenic or pathologic feature of one or more epithelial diseases. The concept of a biomarker also includes a pharmacological or physiological measurement which is used to predict a toxicity event in an animal or a human. A biomarker may be the target for monitoring the outcome of a therapeutic intervention (i.e., the target of a drug agent). In the context of epithelial tissue, by way of example, a biomarker of epithelial disease may be epithelial cell proliferation as measured by the rate of change in DNA synthesis (e.g., a biomarker for various epithelial cancers) or, for example, it may be the rate of keratin synthesis in keratinocytes (e.g., a biomarker for skin epithelial disorders).

By “evaluate” or “evaluation” or “evaluating,” in the context of the present invention, is meant a process whereby the activity, toxicity, relative potency, potential therapeutic value and/or efficacy, significance, or worth of a chemical entity, biological factor, combination of chemical entities, or combination of biological factors is determined through appraisal and study, usually by means of comparing experimental outcomes to established standards and/or conditions. The term embraces the concept of providing sufficient information for a decision-maker to make a “go/no go” decision on a chemical entity or biological factor (or combinations of chemical entities or combinations of biological factors) to proceed further in the drug development process. A “go/no go” decision may be made at any point or milestone in the drug development process including, but not limited to, any stage within pre-clinical development, the pre-clinical to Investigational New Drug (IND) stage, the Phase I to Phase II stage, the Phase II to more advanced phases within Phase II (such as Phase IIb), the Phase II to Phase III stage, the Phase III to the New Drug Application (NDA) or Biologics License Application (BLA) stage, or stages beyond (such as Phase IV or other post-NDA or post-BLA stages). The term also embraces the concept of providing sufficient information to select “best-in-breed” (or “best-of-breed”) in a class of compounds (chemical entities, biologics).

By “characterize,” “characterizing,” or “characterization,” in the context of the present invention is meant an effort to describe the character or quality of a chemical entity or biological factor or combination of chemical entities or combination of biological factors (or mixtures thereof). As used herein, the term is nearly equivalent to “evaluate,” yet lacks the more refined aspects of “evaluate,” in which to “evaluate” a drug includes the ability to make a “go/no go” decision (based on an assessment of therapeutic value) on proceeding with that drug or chemical entity or biological factor through the drug development process.

By “condition” or “medical condition” is meant the physical status of the body as a whole or of one of its parts. The term is usually used to indicate a change from a previous physical or mental status, or an abnormality not recognized by medical authorities as a disease or disorder. Examples of “conditions” or “medical conditions” include, but are not limited to, obesity and pregnancy.

By “therapeutic effect” is meant any effect elicited by a compound or combination of compounds or mixtures of compounds that provides ameliorative or palliative results, or improves, even to the slightest degree, any clinical sign or symptom of an epithelial disease or condition.

By “epithelial tissue toxicity” is meant any toxic effect on a tissue or cell of epithelial origin elicited in response to exposure to a compound or compounds or combination or mixture of compounds. The effect, for example, may manifest itself in such a way as to be detectable by the methods of the present invention and, in that regard is considered a “biomarker of epithelial tissue toxicity.” Examples of biomarkers of epithelial tissue toxicity include, but are not limited to, breast epithelial cell proliferation, colon epithelial cell proliferation, prostate epithelial cell proliferation, ovarian epithelial cell proliferation, endometrial cell proliferation, bronchial epithelial cell proliferation, pancreatic epithelial cell proliferation, keratin synthesis in skin, and keratinocyte proliferation. Epithelial cell proliferation may be measured in a variety of ways including, but not limited to, DNA synthesis or increased synthesis of any protein or protein complex in the cell required for mitosis or cytokinesis. See details, infra.

IV. Methods of the Invention

A. Overview of the Methods of the Invention

The present invention is directed to methods of measuring biomarkers of epithelial disease states, said methods being generally applicable, non-invasive (or minimally invasive), and useful in evaluating or characterizing risk, activity, progression, pathogenesis, mechanism, or therapeutic response for diseases of epithelial tissues, including epithelial cancers. The present invention discloses methods that embody the discovery that (1) epithelial cells (often intact) or important biochemical components (molecules) from epithelial cells can be isolated from excreta; (2) the isotopic content or pattern of these cells or molecules isolated from excreta can be measured using analytic procedures that have been developed by the Applicant or that are known in the art, after administration in vivo to the individual of targeted, isotopically perturbed metabolic precursors, prior to collection of excreta; and (3) the isotopic content or pattern of these cells or molecules isolated from excreta can reveal key information about cell proliferation or other biochemical events (e.g., protein turnover) that characterize the early phase of epithelial carcinogenesis or that are involved in other disorders of epithelial tissues.

By combining the discoveries, supra, into a single procedure (see FIG. 1), the Applicant herein has disclosed a generally applicable, non-invasive (or minimally invasive) method for generating information about diseases of epithelial tissue, including cancer. This combined method thereby fulfills the need for non-invasive or minimally invasive biomarkers of disease risk, activity, progression, pathogenesis, mechanism, or therapeutic response for diseases of epithelial tissues, including epithelial cancers.

The present invention combines the following elements in a manner that solves the need for non-invasive biomarkers of epithelial diseases such as, inter alia, cancer, psoriasis, ulcerative colitis, primary biliary cirrhosis, benign prostatic hyperplasia, chronic bronchitis, bladder hyperplasia, etc.:
Epithelial tissues communicate with the space outside of the body, so that their excreta (luminal or integumental) is often accessible to collection or sampling, by non-invasive or minimally invasive techniques
Excreta from epithelial tissues (luminal or integumental) often contains epithelial cells or molecules released from epithelial cells
   In vivo labeling of epithelial cell biochemical processes (e.g., as DNA replication and cell proliferation) can reveal important and even critical information relevant to the initiation, progression, activity, severity, regression, pathogenesis, mechanism, or therapeutic response of a disease of the epithelial tissue of interest, that is ascertainable by measurements of the isotopic content and/or pattern of epithelial cells or molecules of interest from epithelial cells.

At least one isotope-labeled substrate molecule is administered to a cell, tissue or organism for a period of time sufficient to be incorporated in vivo into one or more molecules of interest within one or more targeted metabolic pathways. In one embodiment, the isotope-labeled substrate molecules are labeled with a stable isotope (i.e., non-radioactive isotope). In another embodiment, the isotope-labeled substrate molecule is labeled with a radioactive isotope. In yet another embodiment, both stable and radioactive isotopes are used to label one or more isotope-labeled substrate molecules.

The targeted molecule of interest is obtained by biochemical isolation procedures from the cell, tissue, or organism, and is identified by mass spectrometry or by other means known in the art. The relative and absolute abundances of the ions within the mass isotopomeric envelope corresponding to each identified molecule of interest (i.e., the isotopic content and/or pattern of the molecule or the rate of change of the isotopic content and/or pattern of the molecule) are quantified. In one embodiment, the relative and absolute abundances of the ions within the mass isotopomeric envelope corresponding to each identified molecule of interest are quantified by mass spectrometry. Flux rates through the targeted metabolic pathways are then calculated by use of equations known in the art and discussed, infra. Flux rates through the targeted metabolic pathways are compared in the presence or absence of exposure to one or more chemical entities or one or more biological factors (i.e., drugs, drug candidates, industrial chemicals, food additives, environmental pollutants, monoclonal or polyclonal antibodies, recombinant proteins, vaccines, and the like) or combinations of chemical entities or combinations of biological factors (i.e., combinations of drugs, drug candidates, or other chemicals, or combinations of biological factors), or in response to different levels of exposure to one or more chemical entities or one or more biological factors, or in response to different levels of exposure to combinations of chemical entities or combinations of biological factors.

In this manner, changes in the targeted underlying biochemical (metabolic) pathways are measured and quantified and related to epithelial disease diagnosis; epithelial disease prognosis; therapeutic efficacy of administered drugs, drug candidates, drug leads, or biological factors; or toxic effects of chemical entities or biological factors such as drug candidates, drug leads, known drugs, industrial chemicals, pesticides, herbicides, cosmetics, food additives, monoclonal or polyclonal antibodies, recombinant proteins, vaccines, and the like.

B. Administering Isotope-Labeled Metabolic Precursor(s)

As a first step in the methods of the invention, isotope-labeled precursors are administered.

1. Administering an Isotope-Labeled Metabolic Precursor Molecule

Modes of administering the one or more isotope-labeled substrates may vary, depending upon the absorptive properties of the isotope-labeled substrate and the specific biosynthetic pool into which each compound is targeted. Precursors may be administered to animals including humans directly for in vivo analysis. In addition, precursors may be administered in vitro to living cells. Specific types of living cells include hepatocytes, adipocytes, myocytes, fibroblasts, neurons, pancreatic β-cells, intestinal epithelial cells, leukocytes, lymphocytes, erythrocytes, microbial cells and any other cell-type that can be maintained alive and functional in vitro.

Generally, an appropriate mode of administration is one that produces a steady state level of precursor within the biosynthetic pool and/or in a reservoir supplying such a pool for at least a transient period of time. Intravascular or oral routes of administration are commonly used to administer such precursors to organisms, including humans. Other routes of administration, such as subcutaneous or intra-muscular administration, optionally when used in conjunction with slow release precursor compositions, are also appropriate. Compositions for injection are generally prepared in sterile pharmaceutical excipients.

a. Labeled Precursor Molecules

(1) Isotope Labels

The first step in measuring molecular flux rates involves administering an isotope-labeled precursor molecule to a cell, tissue, or organism. The isotope labeled precursor molecule may contain a stable isotope or a radioisotope. Isotope labels that can be used in accordance with the methods of the present invention include, but are not limited to, ²H, ¹³C, ¹⁵N, ¹⁸O, ³H, ¹⁴C, ³⁵S, ³²P, ³³P, ¹²⁵I, ¹³¹I, or other isotopes of elements present in organic systems. These isotopes, and others, are suitable for all classes of chemicals (i.e., precursor molecules) envisioned for use in the present invention. Such precursor molecules include, but are not limited to, protein precursors, lipid precursors, carbohydrate precursors, nucleic acid precursors, porphyrin precursors, glycosaminoglycan precursors, and proteoglycan precursors (see examples of each, infra).

In one embodiment, the isotope label is ²H.

(2) Precursor Molecules (Isotope-Labeled Substrates)

The precursor molecule may be any molecule having an isotope label that is incorporated into a molecule of interest by passage through a metabolic pathway in vivo in a living system. Precursor molecules typically used include, without limitation: H₂O; CO₂; NH₃; acetyl CoA (to form cholesterol, fatty acids); ribonucleic acids (to form RNA); deoxyribonucleic acids (to form DNA); glucose (to form glycogen); amino acids (to form peptides/proteins); phosphoenol-pyruvate (to form glucose/UDP-glucose); and glycine/succinate (to form porphyrin derivatives). Isotope labels may be used to modify all precursor molecules disclosed herein to form isotope-labeled precursor molecules.

The entire precursor molecule may be incorporated into one or more molecules of interest within a metabolic pathway. Alternatively, a portion of the precursor molecule may be incorporated into one or more molecules of interest.

i. Protein Precursors

A protein precursor molecule may be any protein precursor molecule known in the art. These precursor molecules may be amino acids, CO₂, NH₃, glucose, lactate, H₂O, acetate, and fatty acids.

The isotope label may include specific heavy isotopes of elements present in biomolecules, such as ²H, ¹³C, ¹⁵N, ¹⁸O, ³³S, ³⁴S, or may contain other isotopes of elements present in biomolecules such as ³H, ¹⁴C, ³⁵S, ³²P, ³³P, ¹²⁵I, or ¹³¹I.

Precursor molecules of proteins may also include one or more amino acids. The precursor may be any amino acid. The precursor molecule may be a singly or multiply deuterated amino acid. The precursor molecule may be one or more of ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. By way of example, isotope labeled protein precursors include, but are not limited to ²H₂O, ¹⁵NH₃, ¹³CO₂, H¹³CO₃, ²H-labeled amino acids, ¹³C labeled amino acids, ¹⁵N labeled amino acids, ¹⁸O labeled amino acids, ³³S or ³⁴S labeled amino acids, ³H₂O, ³H-labeled amino acids, and ¹⁴C labeled amino acids. Labeled amino acids may be administered, for example, undiluted or diluted with non-labeled amino acids. All isotope labeled precursors may be purchased commercially, for example, from Cambridge Isotope Labs (Andover, Mass.).

Protein precursor molecules may also include any precursor for post-translational or pre-translationally modified amino acids. These precursors include but are not limited to precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; and other pre or post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂O in body water may be identified. The total number of C—H bonds in each non essential amino acid is known—e.g., 4 in alanine, 2 in glycine, etc.

The precursor molecule for proteins may be water. The hydrogen atoms on C—H bonds are the hydrogen atoms on amino acids that are useful for measuring protein synthesis from ²H₂O since the O—H and N—H bonds of proteins are labile in aqueous solution. As such, the exchange of ²H-label from ²H₂O into O—H or N—H bonds occurs without the synthesis of proteins from free amino acids as described above. C—H bonds undergo incorporation from H₂O into free amino acids during specific enzyme-catalyzed intermediary metabolic reactions. The presence of ²H-label in C—H bonds of protein-bound amino acids after ²H₂O administration therefore means that the protein was assembled from amino acids that were in the free form during the period of ²H₂O exposure—i.e., that the protein is newly synthesized. Analytically, the amino acid derivative used must contain all the C—H bonds but must remove all potentially contaminating N—H and O—H bonds.

Hydrogen atoms from body water may be incorporated into free amino acids. ²H or ³H from labeled water can enter into free amino acids in the cell through the reactions of intermediary metabolism, but ²H or ³H cannot enter into amino acids that are present in peptide bonds or that are bound to transfer RNA. Free essential amino acids may incorporate a single hydrogen atom from body water into the α-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins.

One of skill in the art will recognize that labeled hydrogen atoms from body water may be incorporated into other amino acids via other biochemical pathways. For example, it is known in the art that hydrogen atoms from water may be incorporated into glutamate via synthesis of the precursor α-ketoglutarate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histidine, the hydroxyl group in hydroxyproline or hydroxylysine, and others. Other amino acid synthesis pathways are known to those of skill in the art.

Oxygen atoms (H₂¹⁸O) may also be incorporated into amino acids through enzyme-catalyzed reactions. For example, oxygen exchange into the carboxylic acid moiety of amino acids may occur during enzyme catalyzed reactions. Incorporation of labeled oxygen into amino acids is known to one of skill in the art. Oxygen atoms may also be incorporated into amino acids from ¹⁸O₂ through enzyme catalyzed reactions (including hydroxyproline, hydroxylysine or other post-translationally modified amino acids).

Hydrogen and oxygen labels from labeled water may also be incorporated into amino acids through post-translational modifications. In one embodiment, the post-translational modification may already include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange labeled hydrogens from body water, either before or after post-translational modification step (e.g., methylation, hydroxylation, phosphorylation, prenylation, sulfation, carboxylation, acetylation or other known post-translational modifications).

Protein precursors that are suitable for administration into a subject include, but are not limited to H₂O, CO₂, NH₃ and HCO₃, in addition to the standard amino acids found in proteins.

The individual being administered a labeled protein precursor may be a mammal. In one variation, the individual may be an experimental animal including, without limitation, a rodent, primate, hamster, guinea pig, dog, or pig. In variations involving the administering of drugs, drug candidates, drug leads, biological factors, or combinations thereof, the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

ii. Precursors of Organic Metabolites

Precursors of organic metabolites may be any precursor molecule capable of entering into the organic metabolite pathway. Organic metabolites and organic metabolite precursors include, but are not limited to, H₂O, CO₂, NH₃, HCO₃, amino acids, monosaccharides, carbohydrates, lipids, fatty acids, nucleic acids, glycolytic intermediates, acetic acid, and tricarboxylic acid cycle intermediates.

Isotope labeled organic metabolite precursors include, but are not limited to, ²H₂O, ¹⁵NH₃, ¹³CO₂, H¹³CO₃, ²H-labeled amino acids, ¹³C-labeled amino acids, ¹⁵N-labeled amino acids, ¹⁸O-labeled amino acids, ³³S or ³⁴S-labeled amino acids, ³H₂O, ³H-labeled amino acids, ¹⁴C-labeled amino acids, ¹⁴CO₂, and H¹⁴CO₂.

Organic metabolite precursors may also be administered directly. Mass isotopes that may be useful in mass isotope labeling of organic metabolite precursors include, but are not limited to, ²H, ³H, ¹³C, ¹⁴C, ¹⁵N, ¹⁸O, ³³S ³⁴S, ³⁵S, ³²P, ¹²⁵I, ¹³¹I, or other isotopes of elements present in organic systems. It is often desirable, in order to avoid metabolic loss of isotope labels, that the isotope-labeled atom(s) be relatively non-labile or at least behave in a predictable manner within the subject. By administering the isotope-labeled precursors to the biosynthetic pool, the isotope-labeled precursors can become directly incorporated into organic metabolites formed in the pool.

The individual being administered a labeled organic metabolite precursor may be a mammal. In one variation, the individual may be an experimental animal including, without limitation, a rodent, primate, hamster, guinea pig, dog, or pig. In variations involving the administering of drugs, drug candidates, drug leads, biological factors, or combinations thereof, the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

iii. Precursors of Nucleic Acids

Precursors of nucleic acids (i.e., RNA, DNA) are any compounds suitable for incorporation into RNA and/or DNA synthetic pathways. Examples of substrates useful in labeling the deoxyribose ring of DNA include, but are not limited to, [6,6-²H₂] glucose,[U-¹³C₆] glucose and [2-¹³C₁] glycerol (see U.S. Pat. No. 6,461,806, herein incorporated by reference). Labeling of the deoxyribose is superior to labeling of the information-carrying nitrogen bases in DNA because it avoids variable dilution sources. The stable isotope labels are readily detectable by mass spectrometric techniques.

In one embodiment, a stable isotope label is used to label the deoxyribose ring of DNA from glucose, precursors of glucose-6-phosphate or precursors of ribose-5-phosphate. In embodiments where glucose is used as the starting material, suitable labels include, but are not limited to, deuterium-labeled glucose such as [6,6-²H₂] glucose, [1-²H₁] glucose, [3-²H₁] glucose, [²H₇] glucose, and the like; ¹³C-1 labeled glucose such as [1-¹³C₁] glucose, [U-¹³C₆] glucose and the like; and ¹⁸O-labeled glucose such as [1-¹⁸O₂] glucose and the like.

In embodiments where a glucose-6-phosphate precursor or a ribose-5-phosphate precursor is desired, a gluconeogenic precursor or a metabolite capable of being converted to glucose-6-phosphate or ribose-5-phosphate can be used. Gluconeogenic precursors include, but are not limited to, ¹³C-labeled glycerol such as [2-¹³C₁] glycerol and the like, a ¹³C-labeled amino acid, deuterated water (²H₂O) and ¹³C-labeled lactate, alanine, pyruvate, propionate or other non-amino acid precursors for gluconeogenesis. Metabolites which are converted to glucose-6-phosphate or ribose-5-phosphate include, but are not limited to, labeled (²H or ¹³C) hexoses such as [1-²H₁] galactose, [U-¹³C] fructose and the like; labeled (²H or ¹³C) pentoses such as [1-¹³C₁] ribose, [1-²H₁] xylitol and the like, labeled (²H or ¹³C) pentose phosphate pathway metabolites such as [1-²H₁] seduheptalose and the like, and labeled (²H or ¹³C) amino sugars such as [U-¹³C] glucosamine, [1-²H₁] N-acetyl-glucosamine and the like.

The present invention also encompasses stable isotope labels which label purine and pyrimidine bases of DNA through the de novo nucleotide synthesis pathway. Various building blocks for endogenous purine synthesis can be used to label purines and they include, but are not limited to, ¹⁵N-labeled amino acids such as [¹⁵N] glycine, [¹⁵N] glutamine, [¹⁵N] aspartate and the like, ¹³C-labeled precursors such as [1-¹³C₁] glycone, [3-¹³C₁] acetate, [¹³C]HCO₃, [¹³C] methionine and the like, and H-labeled precursors such as ²H₂O. Various building blocks for endogenous pyrimidine synthesis can be used to label pyrimidines and they include, but are not limited to, ¹⁵N-labeled amino acids such as [¹⁵N] glutamine and the like, ¹³C-labeled precursors such as [¹³C]HCO₃, [U-¹³C₄] aspartate and the like, and ²H-labeled precursors (²H₂O).

It is understood by those skilled in the art that in addition to the list above, other stable isotope labels which are substrates or precursors for any pathways which result in endogenous labeling of DNA are also encompassed within the scope of the invention. The labels suitable for use in the present invention are generally commercially available or can be synthesized by methods well known in the art.

The individual being administered a labeled nucleic acid precursor may be a mammal. In one variation, the individual may be an experimental animal including, without limitation, a rodent, primate, hamster, guinea pig, dog, or pig. In variations involving the administering of drugs, drug candidates, drug leads, biological factors, or combinations thereof, the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

iv. Water as a Precursor Molecule

Water is a precursor of proteins and many organic metabolites. As such, labeled water may serve as a precursor in the methods taught herein.

H₂O availability is probably never limiting for biosynthetic reactions in a cell (because H₂O represents close to 70% of the content of cells, or >35 Molar concentration), but hydrogen and oxygen atoms from H₂O contribute stochiometrically to many reactions involved in biosynthetic pathways: e.g.: R—CO—CH2-COOH+NADPH+H₂O→R—CH₂CH2COOH (fatty acid synthesis).

As a consequence, isotope labels provided in the form of H- or O-isotope-labeled water is incorporated into biological molecules as part of synthetic pathways. Hydrogen incorporation can occur in two ways: into labile positions in a molecule (i.e., rapidly exchangeable, not requiring enzyme catalyzed reactions) or into stable positions (i.e., not rapidly exchangeable, requiring enzyme catalysis). Oxygen incorporation occurs in stable positions.

Some of the hydrogen-incorporating steps from cellular water into C—H bonds in biological molecules only occur during well-defined enzyme-catalyzed steps in the biosynthetic reaction sequence, and are not labile (exchangeable with solvent water in the tissue) once present in the mature end-product molecules. For example, the C—H bonds on glucose are not exchangeable in solution. In contrast, each of the following C—H positions exchanges with body water during reversal of specific enzymatic reactions: C-1 and C-6, in the oxaloacetate/succinate sequence in the Krebs' cycle and in the lactate/pyruvate reaction; C-2, in the glucose-6-phosphate/fructose-6-phosphate reaction; C-3 and C-4, in the glyceraldehyde-3-phosphate/dihydroxyacetone-phosphate reaction; C-5, in the 3-phosphoglycerate/glyceraldehyde-3-phosphate and glucose-6-phosphate/fructose-6-phosphate reactions.

Labeled hydrogen or oxygen atoms from water that are covalently incorporated into specific non-labile positions of a molecule thereby reveals the molecule's “biosynthetic history”—i.e., label incorporation signifies that the molecule was synthesized during the period that isotope-labeled water was present in cellular water.

The labile hydrogens (non-covalently associated or present in exchangeable covalent bonds) in these biological molecules do not reveal the molecule's biosynthetic history. Labile hydrogen atoms can be easily removed by incubation with unlabelled water (H₂O) (i.e., by reversal of the same non-enzymatic exchange reactions through which ²H or ³H was incorporated in the first place), however:

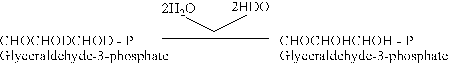

As a consequence, potentially contaminating hydrogen label that does not reflect biosynthetic history, but is incorporated via non-synthetic exchange reactions, can easily be removed in practice by incubation with natural abundance H₂O.

Analytic methods are available for measuring quantitatively the incorporation of labeled hydrogen atoms into biological molecules (e.g., liquid scintillation counting for ³H; mass spectrometry or NMR spectroscopy for ²H and ¹⁸O). For further discussions on the theory of isotope-labeled water incorporation, see, for example, Jungas R L. Biochemistry. 1968 7:3708-17, incorporated herein by reference.

Labeled water may be readily obtained commercially. For example, ²H₂O may be purchased from Cambridge Isotope Labs (Andover, Mass.), and ³H₂O may be purchased, e.g., from New England Nuclear, Inc. In general, ²H₂O is non-radioactive and thus, presents fewer toxicity concerns than radioactive ³H₂O. ²H₂O may be administered, for example, as a percent of total body water, e.g., 1% of total body water consumed (e.g., for 3 litres water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved relatively inexpensively using the techniques of the invention. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of ²H₂O. The Applicant has discovered that as long as rapid changes in body water enrichment are prevented (e.g., by initial administration in small, divided doses), high body water enrichments of ²H₂O can be maintained with no toxicities. For example, the low expense of commercially available ²H₂O allows long-term maintenance of enrichments in the 1-5% range at relatively low expense (e.g., calculations reveal a lower cost for 2 months labeling at 2% ²H₂O enrichment, and thus 7-8% enrichment in the alanine precursor pool, than for 12 hours labeling of ²H-leucine at 10% free leucine enrichment, and thus 7-8% enrichment in leucine precursor pool for that period).

Relatively high and relatively constant body water enrichments for administration of H₂¹⁸O may also be accomplished, since the ¹⁸O isotope is not toxic, and does not present a significant health risk as a result.

Isotope-labeled water may be administered via continuous isotope-labeled water administration, discontinuous isotope-labeled water administration, or after single or multiple administration of isotope-labeled water administration. In continuous isotope-labeled water administration, isotope-labeled water is administered to an individual for a period of time sufficient to maintain relatively constant water enrichments over time in the individual. For continuous methods, labeled water is optimally administered for a period of sufficient duration to achieve a steady state concentration (e.g., 3-8 weeks in humans, 1-2 weeks in rodents).

In discontinuous isotope-labeled water administration, an amount of isotope-labeled water is measured and then administered, one or more times, and then the exposure to isotope-labeled water is discontinued and wash-out of isotope-labeled water from body water pool is allowed to occur. The time course of delabeling may then be monitored. Water is optimally administered for a period of sufficient duration to achieve detectable levels in biological molecules.

Isotope-labeled water may be administered to an individual or tissue in various ways known in the art. For example, isotope-labeled water may be administered orally, parenterally, subcutaneously, intravascularly (e.g., intravenously, intraarterially), or intraperitoneally. Several commercial sources of ²H₂O and H₂¹⁸O are available, including Isotec, Inc. (Miamisburg Ohio, and Cambridge Isotopes, Inc. (Andover, Mass.). The isotopic content of isotope labeled water that is administered can range from about 0.001% to about 20% and depends upon the analytic sensitivity of the instrument used to measure the isotopic content of the biological molecules. In one embodiment, 4% ²H₂O in drinking water is orally administered. In another embodiment, a human is administered 50 mL of ²H₂O orally.

The individual being administered labeled water may be a mammal. In one variation, the individual may be an experimental animal including, without limitation, a rodent, primate, hamster, guinea pig, dog, or pig. In variations involving the administering of drugs, drug candidates, drug leads, biological factors, or combinations thereof, the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

v. Precursors of Carbohydrates

Compositions comprising carbohydrates may include monosaccharides, polysaccharides, or other compounds attached to monosaccharides or polysaccharides.

Isotope labels may be incorporated into carbohydrates or carbohydrate derivatives. These include monosaccharides (including, but not limited to, glucose and galactose), amino sugars (such as N-Acetyl-Galactosamine), polysaccharides (such as glycogen), glycoproteins (such as sialic acid) glycolipids (such as galactocerebrosides), glycosaminoglycans (such as hyaluronic acid, chondroitin-sulfate, and heparan-sulfate) by biochemical pathways known in the art.

²H-labeled sugars may be administered to an individual as monosaccharides or as polymers comprising monosaccharide residues. Labeled monosaccharides may be readily obtained commercially (e.g., Cambridge Isotopes, Massachusetts).

Relatively low quantities of compounds comprising ²H-labeled sugars need be administered. Quantities may be on the order of milligrams, 10¹ mg, 10² mg, 10³ mg, 10⁴ mg, 10⁵ mg, or 10⁶ mg. ²H-labeled sugar enrichment may be maintained for weeks or months in humans and in animals without any evidence of toxicity. The lower expense of commercially available labeled monosaccharides, and low quantity that need to be administered, allow maintenance of enrichments at low expense.

In one embodiment, the labeled sugar is glucose. Glucose is metabolized by glycolysis and the citric acid cycle. Glycolysis releases most of the H-atoms from C—H bonds of glucose; oxidation via the citric acid cycle ensures that all H-atoms are released to H₂O. The loss of ³H- or ²H-label by glucose has been used to assess glycolysis, an intracellular metabolic pathway for glucose. Some investigators have used release of ³H from intravenously administered ³H-glucose into ³H₂O as a measure of glycolysis. Release of ²H-glucose into ²H₂O has not been used previously, because of the expectation that the body water pool is too large relative to ²H administration in labeled glucose to achieve measurable 2H2O levels. In a further variation, the labeled glucose may be [6,6-²H₂]glucose, [1-²H₁]glucose, and [1,2,3,4,5,6-²H₇]glucose.

In another embodiment, labeled sugar comprises fructose or galactose. Fructose enters glycolysis via the fructose 1-phosphate pathway, and secondarily through phosphorylation to fructose 6-phosphate by hexokinase. Galactose enters glycolysis via the galactose to glucose interconversion pathway.

Any other sugar is envisioned for use in the present invention. Contemplated monosaccharides include, but are not limited to, trioses, pentoses, hexoses, and higher order monosaccharides. Monosaccharides further include, but are not limited to, aldoses and ketoses.

In another embodiment, polymers comprising polysaccharides may be administered. In yet another embodiment, labeled polysaccharides may be administered. In yet another embodiment, labeled sugar monomers may be administered as a component of sucrose (glucose α-(1,2)-fructose), lactose (galactose β-(1,4)-glucose), maltose (glucose α-(1,4)-glucose), starch (glucose polymer), or other polymers.

In one embodiment, the labeled sugar may be administered orally, by gavage, intraperitoneally, intravascularly (e.g., intravenously, intraarterially), subcutaneously, or other bodily routes. In particular, the sugars may be administered to an individual orally, optionally as part of a food or drink.

The individual being administered a labeled carbohydrate precursor may be a mammal. In one variation, the individual may be an experimental animal including, without limitation, a rodent, primate, hamster, guinea pig, dog, or pig. In variations involving the administering of drugs, drug candidates, drug leads, biological factors, or combinations thereof, the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

vi. Precursors of Lipids and Other Fats

Measuring the metabolism of compounds comprising ²H-labeled fatty acids are also contemplated by the present invention. Isotope labels from isotope-labeled water may also be incorporated into fatty acids, the glycerol moiety of acyl-glycerols (including but not limited to, triacylglycerides, phospholipids, and cardiolipin), cholesterol and its derivatives (including but not limited to cholesterol-esters, bile acids, steroid hormones) by biochemical pathways known in the art.

Complex lipids, such as glycolipids and cerebrosides, can also be labeled from isotope-labeled water, which is a precursor for the sugar-moiety of cerebrosides (including, but not limited to, N-acetylgalactosamine, N-acetylglucosamine-sulfate, glucuronic acid, and glucuronic acid-sulfate).

²H-labeled fatty acids may be administered to an individual as fats or other compounds containing the labeled fatty acids. ²H-labeled fatty acids may be readily obtained commercially. Relatively low quantities of labeled fatty acids need be administered. Quantities may be on the order of milligrams, 10¹ mg, 10² mg, 10³ mg, 10⁴ mg, 10⁵ mg, or 10⁶ mg. Fatty acid enrichment, particularly with ²H, may be maintained for weeks or months in humans and in animals without any evidence of toxicity. The lower expense of commercially available labeled fatty acids, and low quantity that need to be administered, allow maintenance of enrichments at low expense.

The release of labeled fatty acids, particularly ²H-fatty acid, to labeled water, particularly ²H₂O, accurately reflects fat oxidation. Administration of modest amounts of labeled-fatty acid is sufficient to measure release of labeled hydrogen or oxygen to water. In particular, administration of modest amounts of ²H-fatty acid is sufficient to measure release of ²H to deuterated water.

In another variation, the labeled fatty acids may be administered orally, by gavage, intraperitoneally, intravascularly (e.g., intravenously, intraarterially), subcutaneously, or other bodily routes. In particular, the labeled fatty acids may be administered to an individual orally, optionally as part of a food or drink.

The individual being administered labeled lipid precursors may be a mammal. In one variation, the individual may be an experimental animal including, without limitation, a rodent, primate, hamster, guinea pig, dog, or pig. In variations involving the administering of drugs, drug candidates, drug leads, biological factors, or combinations thereof, the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

C. Obtaining One or More Targeted Molecules of Interest

In practicing the methods of the invention, in one aspect, targeted molecules of interest are obtained from a cell, tissue, or organism according to methods known in the art. The methods may be specific to the particular molecule of interest. Molecules of interest may be isolated from a biological sample, said sample derived from material, solid or liquid, obtained from an external space (e.g., from within a luminal or integumentary space).

A plurality of molecules of interest may be acquired from the cell, tissue, or organism. The one or more biological samples may be obtained from spaces external to the body, for example, by urine collection, semen collection, vaginal secretion collection, saliva collection, lacrimal fluid collection, breast ductal fluid collection, sputum collection, gastrointestinal secretion collection, sweat, feces, flatulence, expired air, skin scrapings, skin flakes, or other minimally or non-invasive methods known in the art. The one or more biological sample may be one or more biological fluids. Molecules of interest also may be obtained, and optionally partially purified or isolated, from the biological sample using standard biochemical methods known in the art.

The frequency of biological sampling can vary depending on different factors. Such factors include, but are not limited to, the nature of the molecules of interest, ease and safety of sampling, synthesis and breakdown/removal rates of the molecules of interest, and the half-life of a compound (chemical entity, biological factor, already-approved drug, drug candidate, drug lead, etc.).

The molecules of interest may also be purified partially, or optionally, isolated, by conventional purification methods including high pressure liquid chromatography (HPLC), fast performance liquid chromatography (FPLC), chemical extraction, thin layer chromatography, gas chromatography, gel electrophoresis, and/or other separation methods known to those skilled in the art.

In another embodiment, the molecules of interest may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase degradation). Hydrolysis or degradation may be conducted either before or after purification and/or isolation of the molecules of interest. The molecules of interest may also be partially purified, or optionally, isolated, by conventional purification methods including high performance liquid chromatography (HPLC), fast performance liquid chromatography (FPLC), gas chromatography, gel electrophoresis, and/or any other methods of separating chemical and/or biochemical compounds known to those skilled in the art.

Molecules of interest also include DNA from prostate epithelial cells, DNA from breast epithelial cells, DNA from colon epithelial cells, keratin from skin, hair, or fingernails, and mucin from the intestinal lumen. Furthermore, as molecules of interest in this application are sometimes isolated from excreta, such as stool, that are biologically active (i.e., metabolic processes occur within the excreta, an example being the metabolic processing of excreta by bacteria in stool), molecules of interest may actually be the metabolic products of these biological activities, which may convert the initial labeled molecule of interest into a different, but still labeled, biomolecule.

D. Analysis

Presently available technologies (static methods) used to identify biological actions of agents measure only composition, structure, or concentrations of molecules in a cell and do so at one point in time. Using RNA and protein by way of example, RNA and protein expression “chips can be used to detect multiple biological molecules at one time in cells or organisms in a variety of disease states, although these techniques fail to determine the molecular flux rates of proteins or mRNA transcripts. The methods of the present invention allow determination of the molecular flux rates of a plurality of proteins or transcripts, as well as the molecular flux rates of a plurality of organic metabolites, lipids, DNA, and the like, and their changes over time in a variety of disease states and in response to exposure to one or more drugs, drug candidates, drug leads, biological factors, or combinations thereof, or in response to exposure to one or more industrial chemicals, food additives, cosmetics, or environmental pollutants, or as a measure of disease progression, pathology, and the like.

1. Mass Spectrometry

Mass spectrometers convert components of a sample into rapidly moving gaseous ions and separate them on the basis of their mass-to-charge ratios. The distributions of isotopes or isotopologues of ions, or ion fragments, may thus be used to measure the isotopic enrichment in one or more molecules of interest.

Generally, mass spectrometers include an ionization means and a mass analyzer. A number of different types of mass analyzers are known in the art. These include, but are not limited to, magnetic sector analyzers, electrostatic analyzers, quadrupoles, ion traps, time of flight mass analyzers, and fourier transform analyzers. In addition, two or more mass analyzers may be coupled (MS/MS) first to separate precursor ions, then to separate and measure gas phase fragment ions.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS is used to measure mass isotopomer abundances of organic molecules, hydrogen-labeled isotope incorporation from labeled water is amplified 3 to 7-fold, depending on the number of hydrogen atoms incorporated into the organic molecule from labeled water.

In one embodiment, isotope enrichments of molecules of interest may be measured directly by mass spectrometry.

In another embodiment, the molecules of interest may be partially purified, or optionally isolated, prior to mass spectral analysis. Furthermore, hydrolysis or degradation products of molecules of interest may be purified.

In another embodiment, isotope enrichments of molecules of interest after hydrolysis of the molecule of interest are measured by gas chromatography-mass spectrometry.

In each of the above embodiments the biosynthesis rate of the biological molecule (i.e., molecule of interest) can be calculated by application of the precursor-product relationship (discussed further, infra) using either labeled precursor molecule enrichment values or asymptotic isotope enrichment of a fully turned over molecule of interest to represent the true precursor pool enrichment. Alternatively, the biosynthesis or breakdown rate may be calculated using an exponential decay curve by application of exponential or other die-away kinetic models (discussed further, infra).

a. Measuring Relative and Absolute Mass Isotopomer Abundances

Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the present invention.

2. Calculating Labeled: Unlabeled Proportion of Molecules of Interest

The proportion of labeled and unlabeled molecules of interest is then calculated. The practitioner first determines measured excess molar ratios for isolated isotopomer species of a molecule. The practitioner then compares measured internal pattern of excess ratios to the theoretical patterns. Such theoretical patterns can be calculated using the binomial or multinomial distribution relationships as described in U.S. Pat. Nos. 5,338,686, 5,910,403, and 6,010,846, which are hereby incorporated by reference in their entirety. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California, Berkeley.

The comparison of excess molar ratios to the theoretical patterns can be carried out using a table generated for a molecule of interest, or graphically, using determined relationships. From these comparisons, a value, such as the value p, is determined, which describes the probability of mass isotopic enrichment of a subunit in a precursor subunit pool. This enrichment is then used to determine a value, such as the value A_{X}*, which describes the enrichment of newly synthesized proteins for each mass isotopomer, to reveal the isotopomer excess ratio which would be expected to be present, if all isotopomers were newly synthesized.

Fractional abundances are then calculated. Fractional abundances of individual isotopes (for elements) or mass isotopomers (for molecules) are the fraction of the total abundance represented by that particular isotope or mass isotopomer. This is distinguished from relative abundance, wherein the most abundant species is given the value 100 and all other species are normalized relative to 100 and expressed as percent relative abundance. For a mass isotopomer M_{X},

$$Fractional abundance of {M}_{X} = {A}_{X} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

$$Δ Fractional abundance ( enrichment or depletion ) = \\ {( {A}_{x} )}_{e} - {( {A}_{x} )}_{b} = {( \frac{Abundance {M}_{x}}{{\sum }_{i = 0}^{n} Abundance {M}_{i}} )}_{e} - {( \frac{Abundance {M}_{x}}{{\sum }_{i = 0}^{n} Abundance {M}_{i}} )}_{b} ,$$

where subscript e refers to enriched and b refers to baseline or natural abundance.

In order to determine the fraction of polymers that were actually newly synthesized during a period of precursor administration, the measured excess molar ratio (EM_{X}) is compared to the calculated enrichment value, A_{X}*, which describes the enrichment of newly synthesized biopolymers for each mass isotopomer, to reveal the isotopomer excess ratio which would be expected to be present, if all isotopomers were newly synthesized.

3. Calculating Molecular Flux Rates

The method of determining rate of synthesis includes calculating the proportion of mass isotopically labeled subunit present in the molecular precursor pool, and using this proportion to calculate an expected frequency of a molecule of interest containing at least one mass isotopically labeled subunit. This expected frequency is then compared to the actual, experimentally determined isotopomer frequency of the molecule of interest. From these values, the proportion of the molecule of interest which is synthesized from added isotopically labeled precursors during a selected incorporation period can be determined. Thus, the rate of synthesis during such a time period is also determined.

A precursor-product relationship may then be applied. For the continuous labeling method, the isotopic enrichment is compared to asymptotic (i.e., maximal possible) enrichment and kinetic parameters (e.g., synthesis rates) are calculated from precursor-product equations. The fractional synthesis rate (kₛ) may be determined by applying the continuous labeling, precursor-product formula:
kₛ=[−In(1−f)]/t,

where f=fractional synthesis=product enrichment/asymptotic precursor/enrichment

and t=time of label administration of contacting in the system studied.

For the discontinuous labeling method, the rate of decline in isotope enrichment is calculated and the kinetic parameters of the molecules of interest are calculated from exponential decay equations. In practicing the method, biopolymers are enriched in mass isotopomers, preferably containing multiple mass isotopically labeled precursors. These higher mass isotopomers of the molecules of interest, e.g., molecules containing 3 or 4 mass isotopically labeled precursors, are formed in negligible amounts in the absence of exogenous precursor, due to the relatively low abundance of natural mass isotopically labeled precursor, but are formed in significant amounts during the period of molecular precursor incorporation. The molecules of interest taken from the cell, tissue, or organism at the sequential time points are analyzed by mass spectrometry, to determine the relative frequencies of a high mass isotopomer. Since the high mass isotopomer is synthesized almost exclusively before the first time point, its decay between the two time points provides a direct measure of the rate of decay of the molecule of interest.

Preferably, the first time point is at least 2-3 hours after administration of precursor has ceased, depending on mode of administration, to ensure that the proportion of mass isotopically labeled subunit has decayed substantially from its highest level following precursor administration. In one embodiment, the following time points are typically 1-4 hours after the first time point, but this timing will depend upon the replacement rate of the biopolymer pool.

The rate of decay of the molecule of interest is determined from the decay curve for the three-isotope molecule of interest. In the present case, where the decay curve is defined by several time points, the decay kinetic can be determined by fitting the curve to an exponential decay curve, and from this, determining a decay constant.

Breakdown rate constants (k_{d}) may be calculated based on an exponential or other kinetic decay curve:
k_{d}=[−In f]/t.

As described, the method can be used to determine subunit pool composition and rates of synthesis and decay for substantially any biopolymer which is formed from two or more identical subunits which can be mass isotopically labeled. Other well-known calculation techniques and experimental labeling or de-labeling approaches can be used (e.g., see Wolfe, R. R. Radioactive and Stable Isotope Tracers in Biomedicine: Principles and Practice of Kinetic Analysis. John Wiley & Sons; (March 1992)) for calculation flux rates of molecules and flux rates through metabolic pathways of interest.

E. Uses of the Methods of the Present Invention

As discussed in IV A, supra, the methods disclosed herein have many biological and medical applications. The measurements described herein are applicable for numerous medical utilities such as monitoring pre-existing physiological conditions, diagnosis of epithelial disease states, and assessing risk of development of epithelial disease states or physiological conditions, in addition to pharmaceutical research utilities, such as screening of candidate gene or protein targets, phenotypic validation of candidate drug agents, FDA phase I and II human validation studies of candidate drug agents, FDA phase III approval of candidate drug agents, and FDA phase IV approval studies, or other post approval market positioning or mechanism of drug action studies. The disclosed methods allow for the collection of labeled epithelial cells or molecules associated with said cells from areas external to the body (i.e., luminal or integumenary areas) collected from solid, liquid, or gaseous samples.

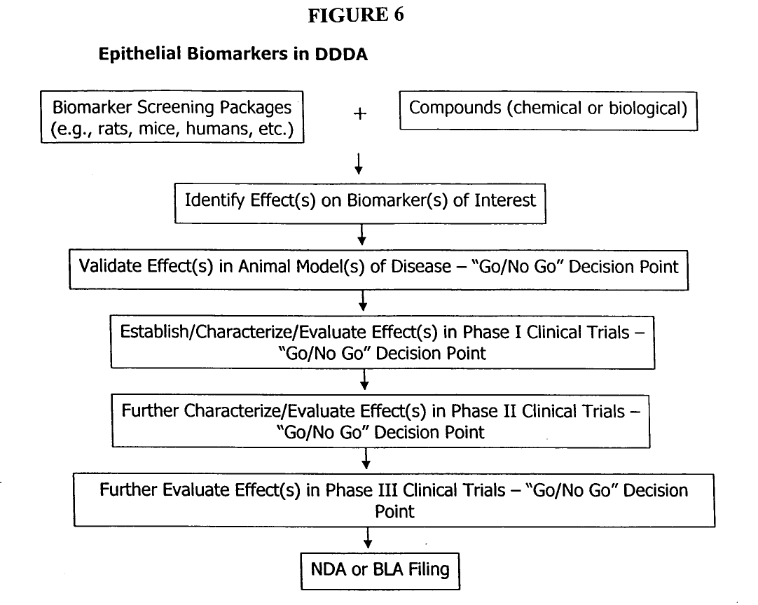

In one embodiment, the methods allow for effects on epithelial biomarkers to be observed after a living system is exposed to a compound or combinations of compounds. The data generated and analyzed is therefore useful in the drug discovery, development, and approval (DDA) process as it facilitates the DDA decision-making process; i.e., it provides useful information for decision-makers in their decision to continue with further development on a compound or combination of compounds (e.g., if the epithelial biomarker data appear promising) or to cease said efforts, for example, if the epithelial biomarker data appear unfavorable (see FIG. 6 for a graphical depiction of this process).

Moreover, the methods allow for the skilled artisan to identify, select, and/or characterize “best in breed” in a class of compounds. Once identified, selected, and/or characterized, the skilled artisan, based on the information generated by the methods of the present invention, can decide to evaluate the “best in breed” further or to license the compound to another entity such as a pharmaceutical company or biotechnology company.

In another embodiment, the methods of the present invention allow for the characterization or evaluation (or both the characterization and evaluation) of toxic effects to epithelial tissues from exposure to industrial chemicals, food additives, cosmetics, and environmental pollutants. The methods of the present invention can be used to establish programs to identify and explore the molecular mechanisms of industrial, food, cosmetic, and environmental toxicants on epithelial tissues to further public health goals.

In one embodiment, data generated by the methods of the present invention may be relevant to understanding an underlying molecular pathogenesis, or causation of, one or more epithelial diseases (e.g., breast cancer or psoriasis). In another embodiment, data generated by the methods of the present invention may shed light on fundamental aspects of the initiation, progression, severity, pathology, aggressiveness, grade, activity, disability, mortality, morbidity, disease sub-classification or other underlying pathogenic or pathologic feature of an epithelial disease of interest.

In yet another embodiment, the data generated by the methods of the present invention may provide elucidation on fundamental aspects of the prognosis, survival, morbidity, mortality, stage, therapeutic response, symptomology, disability or other clinical factor of an epithelial disease of interest. Two or more epithelial biomarkers may be measured independently or concurrently (e.g., DNA synthesis and keratin synthesis).

Biomarkers of epithelial diseases include, but are not limited to, colon epithelial cell proliferation, mammary epithelial cell proliferation, prostate epithelial cell proliferation, ovarian epithelial cell proliferation, endometrial cell proliferation, bronchial epithelial cell proliferation, pancreatic epithelial cell proliferation, keratin synthesis in skin, and keratinocyte proliferation.

Known animal models of epithelial tissue disease may be used as part of the present invention. Such animal models of disease may include, but are not limited to, psoriasis, skin photoaging, skin rashes, breast cancer, prostate cancer, colon cancer, pancreatic cancer, endometrial cancer, endometriosis, and lung cancer.

In another embodiment, the methods of the invention are useful in detecting toxic effects of industrial or occupational chemicals, food additives, cosmetics, or environmental pollutants/contaminants on epithelial tissues. Such toxic effects may include end-organ toxicity. End-organ toxicity may include, but is not limited to, breast epithelial cell proliferation, colon epithelial cell proliferation, prostate epithelial cell proliferation, ovarian epithelial cell proliferation, bronchial epithelial cell proliferation, pancreatic epithelial cell proliferation, keratin synthesis in skin, and keratinocyte proliferation.

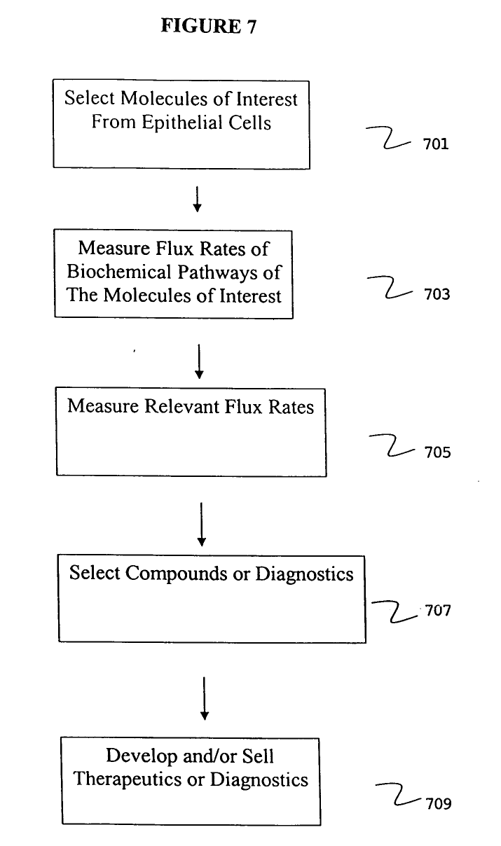

FIG. 7 illustrates the use of the inventions herein in a drug discovery process. At step 701 a plurality of drug candidates or other compounds are selected. At step 703 the flux rates of biomarkers are studied within epithelial cells, preferably according to the methods discussed herein. In alternative embodiments, step 703 is conducted first when the inventions are used, for example, in a target discovery process. At step 705 relevant flux rates are identified. For example, if it is desirable to reduce the flux rate of a particular epithelial biomarker in a particular phenotypic state, a compound that reduces that flux rate will be considered generally more useful, and conversely a compound that increases that flux rate will be considered generally less desirable. In a target discovery process, a particular phenotype that has increased or decreased flux rates with respect to another phenotype (e.g., diseased vs. not diseased) may be considered a good therapeutic or diagnostic target or in the pathway of a good therapeutic or diagnostic target. At step 707 compounds of interest, targets of interest, or diagnostics are selected and further used and further developed. In the case of targets, such targets may be the subject of, for example, well known small molecule screening processes (e.g., high-throughput screening of new chemical entities) and the like. Alternatively, biological factors, or already-approved drugs, or other compounds (or combinations and/or mixtures of compounds) may be used. At step 709 the compounds or diagnostics are sold or distributed. It is recognized of course that one or more of the steps in the process in FIG. 7 will be repeated many times in most cases for optimal results.

F. Isotopically-Perturbed Molecules

In another variation, the methods provide for the production of isotopically-perturbed molecules (e.g., labeled fatty acids, lipids, carbohydrates, proteins, nucleic acids and the like). These isotopically-perturbed molecules comprise information useful in determining the changes in one or more epithelial biomarkers. Once isolated from a cell and/or a tissue of an organism, one or more isotopically-perturbed molecules are analyzed to extract information as described, supra. Such isotopically perturbed molecules include, but are not limited to, DNA from colon epithelial cells, DNA from breast epithelial cells, DNA from colon epithelial cells, mucin from intestinal secretions, and keratin from skin.

The extent of isotopic perturbation may vary depending on the particular type of disease in question and the disease state or treatment regimen. An isotopically perturbed molecule from a very early stage in a disease or from a healthy subject may have an extremely low (potentially undetectable) degree of isotopic perturbation whereas the degree of isotopic perturbation may be high in patients with advanced disease.

G. Kits

The invention also provides kits for measuring changes in epithelial biomarkers in vivo. The kits may include isotope-labeled precursor molecules, and may additionally include chemical compounds known in the art for separating, purifying, or isolating proteins, and/or chemicals necessary to obtain a tissue sample, automated calculation software for combinatorial analysis, and instructions for use of the kit.

Other kit components, such as tools for administration of water (e.g., measuring cup, needles, syringes, pipettes, IV tubing), may optionally be provided in the kit. Similarly, instruments for obtaining samples from the cell, tissue, or organism (e.g., specimen cups, needles, syringes, and tissue sampling devices) may also be optionally provided.

H. Information Storage Devices

The invention also provides for information storage devices such as paper reports or data storage devices comprising data collected from the methods of the present invention. An information storage device includes, but is not limited to, written reports on paper or similar tangible medium, written reports on plastic transparency sheets or microfiche, and data stored on optical or magnetic media (e.g., compact discs, digital video discs, optical discs, magnetic discs, and the like), or computers storing the information whether temporarily or permanently. The data may be at least partially contained within a computer and may be in the form of an electronic mail message or attached to an electronic mail message as a separate electronic file. The data within the information storage devices may be “raw” (i.e., collected but unanalyzed), partially analyzed, or completely analyzed. Data analysis may be by way of computer or some other automated device or may be done manually. The information storage device may be used to download the data onto a separate data storage system (e.g., computer, hand-held computer, and the like) for further analysis or for display or both. Alternatively, the data within the information storage device may be printed onto paper, plastic transparency sheets, or other similar tangible medium for further analysis or for display or both.

The Following References Cited Herein are Incorporated by Reference in their Entirety:

1. Sporn M B. Carcinogenesis and cancer: different perspectives on the same disease. Cancer Res 51:6215-6218, 1991.
2. Kelloff G J, Boone G W, Cromwell J A, et al. Surrogate end-point biomarkers for phase II cancer chemoprevention trials. J Cell Biochem Suppl 19:1-9, 1994.
3. Cohen S M, Ellwein L B. Cell proliferation in carcinogenesis. Science 249:1007-1011, 1990.
4. Miracco C, Pellegrino M, Flori M L, Vatti R, Materno M, Andreassi L. Cyclin D1, B and A expression and cell turnover in psoriatic skin lesions before and after cyclosporine treatment. Br J Dermatol 143:950-956, 2001.
5. Neese R A, Misell L M, Turner S, Chu A, Kim J, Cesar D, Hoh R, Antelo F, Strawford A, McCune J M, Christiansen M, Hellerstein M K. Measurement in vivo of proliferation rates of slow turnover cells by 2H2O labeling of the deoxyribose moiety of DNA. Proc Natl Acad Sci USA 99(24):15345-50, 2002.
6. Hellerstein M, Hoh R, Cesar D, Neese R A, Wieder E, Hanley M B, McCune J M. Kinetic subpopulations of T lymphocytes in humans: role of HIV-1 pathogenesis. J Clin Invest 112(6):956-966, 2003.
7. Fanara P. Turner S, Busch R, Killion S, Awada M, Turner H, Mahsut A, Laprade K L, Stark J M, Hellerstein M K. In vivo measurement of microtubule dynamics using stable isotope labeling with heavy water. Effect of taxanes. J Biol Chem. 2004 November 26;279(48):49940-7.

### I. EXAMPLES

The following non-limiting examples further illustrate the invention disclosed herein:

### Example 1

Isolation of Breast Epithelial Cells from Ductal Lavage Fluid of Women

a) introduction. Breast epithelial cell proliferation is thought to be accelerated as a precursor to the development of breast cancer. Breast epithelial cell (BEC) hyper-proliferation is an example of an initiation or promotional phase phenomenon that occurs prior to the development of clinically detectable cancer (i.e., a phenomenon that is on the pathway to cancer, but occurs before cancer—described in detail, supra). Lavage of the breast ducts can be performed to harvest epithelial cells shedded by the epithelial layer in the ducts—the same cells that are hyperproliferative prior to cancer. In the case of BEC's, the experiments described in this example can be used to differentiate between normal subjects and subjects that have precancerous hyper-proliferative BEC's. In human subjects, in a clinical scenario, the finding of BEC hyper-proliferation may indicate a need for cancer preventative or cancer therapy (if available), or may suggest that more frequent cancer screening may be appropriate. Similarly, in a clinical tria/scenario, such data may provide information on whether or not a cancer-preventative or cancer therapy is effective. In animal models, similar such data can be used for the pre-clinical evaluation of potential therapeutics.

b) methods. Healthy women drank a dose of 50-100 ml/day of heavy water (deuterated water, ²H₂O) for 7-14 days. The women then underwent a ductal lavage procedure, and a blood sample was also collected. Breast epithelial cells were isolated from other cell types in the ductal lavage specimens by an immunomagnetic bead method. Purity of BEC's isolated by this method was >95%. Blood monocytes, which represent a 100% turned over cell population, were also isolated from blood by immunomagnetic bead method and used as a comparison tissue for calculating percent new cells (because they proliferate so rapidly, these cells provide a measure of the maximum possible level of deuterium incorporation into DNA of newly divided cells, and are used to correct for variability in body water deuterium levels, such as those arising from partial compliance with the deuterated water regimen by subjects).

Isolated BEC's and monocytes were lysed and DNA was isolated using a Qiagen Qiamp DNA isolation kit per protocols provided by the manufacturer. Isolated DNA was then enzymatically hydrolyzed. The dA and dG were converted to deoxyribose (dR) and derivatized to pentafluorobenzyloxime-deoxyribose (PFTA) by the following procedures: Acetic acid and perfluorobenzylhydroxylamine (PFBHA) were added to the DNA hydrosylate and the sample was heated at 100° C. for 30 min. Acetic anhydride and 1-methylimidazole were added and sample was incubated at room temperature for 15 min. The reaction mixture was added to water and the derivative was extracted with dichloromethane. An Agilent model 5973 MS with 6890N Network GC and autosampler (Agilent, Palo Alto, Calif.) was used to analyze ²H enrichment. Abundances of ions at mass to charge ratio (m/z), 435 and 436 are quantified under selected ion recording mode for the PTFA derivative. Background isotopic enrichment, of dA standards run concurrently with samples, was subtracted from sample enrichments to give excess enrichments of M+1 (EM+1). Isotopic enrichments (EM+1) of BEC were divided by monocyte (representing 100% turned over tissue) EM+1 enrichments from the same subject to determine % new BEC (f, fractional synthesis). Fractional replacement rate constants (k) were calculated as:

$$( k ) = \frac{ln [ 1 - f ]}{t}$$

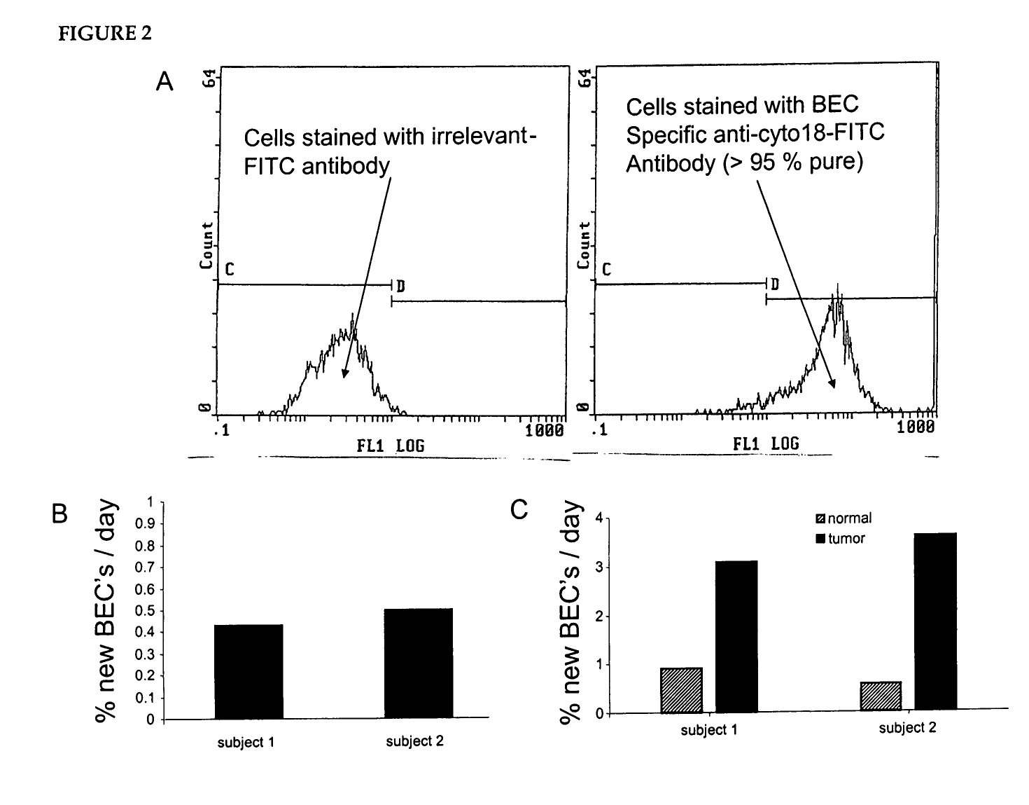

c) results. Calculated proliferation rates of BEC isolated from ductal lavage fluid of women are shown in FIG. 2. In FIG. 2A, verification of the purity of isolated BEC's is shown—more than 95% of the cells isolated stain with a BEC-specific antibody. In FIG. 2B, the measured proliferation rate for both women is shown. Both are normal healthy volunteers, and show a baseline proliferation rate of ˜0.5% new BEC's per day (i.e., every day, about 0.5% of the total BEC's in the subject are newly divided).

d) discussion. The data shown in this example illustrates that BEC proliferation can be easily evaluated using the methods described herein. The utility of this measurement is made apparent by FIG. 2C. In FIG. 2C, BEC proliferation was measured by the same technique, but with BEC's isolated from breast tissue biopsy samples taken from women with breast tumors. In this case, breast epithelial cell proliferation was observed to be much higher in the tumorous tissue versus the non-tumorous tissue. The normal BEC proliferation observed with the ductal lavage samples is the same as that observed in tissue biopsy samples. The data in FIG. 2C illustrates the value of this measurement in measuring the extent of the cancerous or pre-cancerous state. In a clinical trial situation, data collected from samples taken as described in this example could be used to measure the effect of a cancer or cancer-preventative therapy on hyperproliferative BEC's. In such a scenario, an increase in BEC proliferation, like that observed in tumors (FIG. 2C) would indicate a pre-cancerous or cancerous state, and would be expected to decrease when an effective therapy is given. The enormous advantage of the technique described here is that, unlike tissue biopsies, lavage samples are less invasive and can be taken frequently, but still yield the same quality data. Furthermore, because it is less invasive, lavage can be carried out on a broader patient base (because the clinical evidence required to justify the procedure need not be as persuasive if the procedure is less invasive).

While these arguments are made specifically for the example of ductal lavage, they can generally be applied to the other examples presented here—it is reasonable to assume that the collection of excreta or fluids of any sort will be less invasive, less dangerous, less painful, and less risky than direct sampling of the tissue in question.

### Example 2

Isolation of Prostate Epithelial Cells from Seminal Fluid (Ejaculate) from Men

a) introduction. Similar to breast epithelial cell proliferation (Example 1, supra) prostate epithelial cell proliferation is thought to be accelerated as a precursor to the development of prostate cancer. Such prostate epithelial cell (PEC) hyperproliferation may also be a critical event in the development of benign prostate hyperplasia (BPH), a non-malignant syndrome wherein the prostate becomes enlarged. Prostate cancer is a significant cause of mortality among men, and BPH is responsible for a staggering decrease in quality of life in the male population. Prostate epithelial cell hyper-proliferation is an example of an initiation or promotional phase phenomenon that occurs prior to the development of clinically detectable cancer (i.e., a phenomenon that is on the pathway to cancer, but occurs before cancer—described in detail, supra). PEC hyperproliferation may also occur prior to the development of clinically relevant BPH. The prostate provides the bulk of the seminal fluid in ejaculate, and cells shedded by prostate epithelial tissue are found in this fluid. In this case, PEC's can be isolated from ejaculate and used to differentiate between subjects who are normal, and those with pre-cancerous or pre-BPH hyperproliferative PEC's. In human subjects, in a clinical scenario, the finding of PEC hyper-proliferation may indicate a need for cancer preventative or cancer therapy (if available), or may suggest that more frequent cancer screening may be appropriate. It may also be appropriate for the recommendation of BPH suppressive or preventative therapy (if available). Similarly, in a clinical trial scenario, such data may provide information on whether or not a cancer-preventative, cancer, BPH, or BPH preventative therapy is effective. In animal models, similar such data can be used for the pre-clinical evaluation of potential therapeutics.

b) methods. Healthy men drank a 50-100 ml/day dose of heavy water (deuterated water, ²H₂O) for 1-12 weeks. The men provided semen samples (ejaculated semen) weekly. Semen was liquefied by addition of Sperm Prep Viscolytic System (ZDL, Inc.). Sperm cells were then isolated from seminal fluid by Percoll gradient centrifugation. After Percoll gradient removal of the sperm fraction, the epithelial cell containing gradient fraction was subjected to immunomagnetic bead isolation. Seminal fluid prostate epithelial cells (PEC) express surface markers such as prostate specific membrane antigen (PSMA), regardless of disease state of the cells. Immunomagnetic bead isolation of PEC was carried out using a mouse anti-human PSMA primary antibody followed by a goat anti-mouse IgG conjugated immunomagnetic bead. Purity of PEC by intracellular staining of isolated cells with fluorochrome conjugated prostatic alkaline phosphatase (PAP) was 95% by flow cytometry. Monocytes, which represent a 100% turned over cell population, were also isolated from blood and used as a comparison tissue for calculating percent new cells as described previously (5,6). Monocytes were isolated from blood as CD14+ cells by an immunomagnetic bead method. PEC and monocytes were lysed and DNA was isolated using a Qiagen Qiamp DNA isolation kit per protocol. DNA was subjected to enzymatic hydrolysis. The dA and dG were converted to deoxyribose (dR) and derivatized to pentafluorobenzyloxime-deoxyribose (PFTA) by the following procedures: Acetic acid and perfluorobenzylhydroxylamine (PFBHA) were added to the DNA hydrosylate and the sample was heated at 100° C. for 30 min. Acetic anhydride and 1-methylimidazole were added and sample was incubated at room temperature for 15 min. The reaction mixture was added to water and the derivative was extracted with dichloromethane. An Agilent model 5973 MS with 6890N Network GC and autosampler (Agilent, Palo Alto, Calif.) was used to analyze ²H enrichment. Abundances of ions at mass to charge ratio (m/z), 435 and 436 are quantified under selected ion recording mode for the PTFA derivative. Background isotopic enrichment, of dA standards run concurrently with samples, was subtracted from sample enrichments to give excess enrichments of M+1 (EM+1). Isotopic enrichments (EM+1) of BEC were divided by monocyte (representing 100% turned over tissue) EM+1 enrichments from the same subject to determine % new BEC (f, fractional synthesis). Fractional replacement rate constants (k) were calculated as:

$$( k ) = \frac{ln [ 1 - f ]}{t}$$

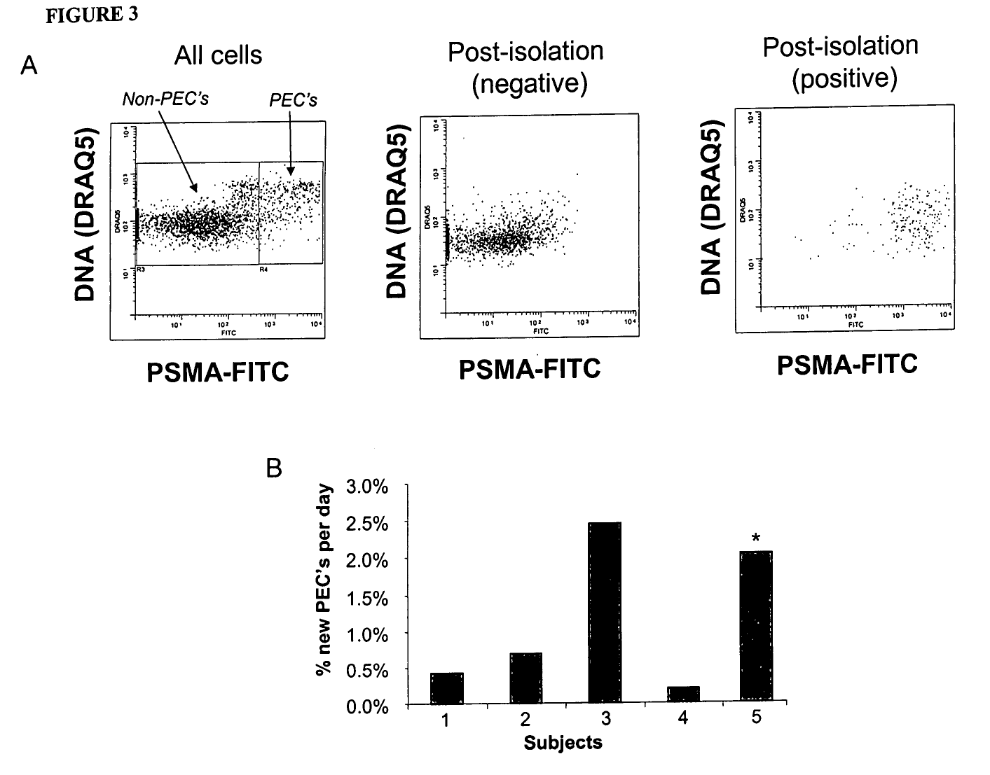

c) results. PEC's were isolated as described, and an analysis of pre and post-isolated cells is shown in FIG. 3A. In FIG. 3A, the left panel shows all of the cells, and the right two panels show either the negative fraction or the positive fraction of flow-sorted (FACS) cells. The rightmost panel shows the isolated PEC's, which are more than 95% pure. Calculated proliferation rates of PEC's isolated from 4 men are shown in FIG. 3B. Patient 3 appears to have elevated PEC proliferation, and may be in a pre-cancerous or pre-BPH state, although further clinical data were not available for this subject. Patients 1, 2, and 4 were considered normal, and did not have any of the urologic or other symptoms associated with prostate cancer or BPH. Patient 5 had an elevated level of PEC proliferation, and was later determined to have BPH. Patient 5 represents an excellent example of the power of this technique—the BPH patient is easily distinguished from other patients.

d) discussion. As with breast epithelial cells (see Example 1, supra), the proliferative behavior of prostate epithelial cells is a clinically relevant parameter that can be used for a variety of purposes. Hyper-proliferative behavior can be an indication of pre-cancer, pre-BPH, cancer, or BPH, and can indicate a need for cancer or BPH treatment or preventative treatment, or a need for additional or more frequent cancer screening. Similarly, a reduction of such hyper-proliferation can be used as evidence for treatment effectiveness. The use of such a technique in a pre-clinical setting for the development of therapies may also be considered. In the case of PEC proliferation, the current invention allows for a quantitative assessment of prostate proliferative behavior. Other measures of prostate health, such as plasma prostate specific antigen (PSA) or prostate exam are either relatively insensitive or inaccurate (PSA) or both qualitative in nature and unpleasant (prostate exam).

### Example 3

Isolation of Keratin from Skin Strips from Rodents and Humans

a) introduction. Maintenance of healthy skin is critical for the existence of most mammals. The skin provides a physical and chemical barrier against infection and dehydration. Because of its barrier function, the skin is constantly flaking off and is replaced by new skin, which derives from an underlying layer of proliferating cells that mature and differentiate as they approach the surface of the body. Mature skin cells are called keratinocytes, and they derive from a basal epidermal layer. As they progress towards the surface of the skin, they synthesize copious amounts of the protein keratin, which accounts for the bulk of the mass of skin cells. During this time, the cells also die. The outer layer of skin is made up of keratin filled dead cells that are constantly shedded and replaced.

Skin hyper-proliferation is associated with a number of disorders. Psoriasis is a skin disorder characterized by painful unattractive scales that cover large tracts of the skin. Skin hyper-proliferation may also be a component of a number of other skin related diseases, such as eczema. Skin proliferation is also a critical component of wound healing, including with respect to recovery from burns, and in the context of diabetic ulcers. Finally, there is considerable interest in skin proliferation and health in the cosmetic and cosmetic surgery industries.

With this in mind, the evaluation of skin proliferation in vivo would allow for the discovery of therapeutics or agents for use in any number of situations, including the treatment of psoriasis, diabetic ulcers, burns, wounds. Further applications are easily considered in the cosmetic and cosmetic surgery industry. Skin proliferation and growth is easily evaluated using the current invention by studying the keratin that makes up the bulk of the skin.

b) methods.

i) Mice. Healthy mice or FSN mice (flaky skin mice—mutant mice with a psoriasis-like phenotype) were given an intraperitoneal injection of ²H₂O to 5% body water content, and then 8% ²H₂O as drinking water for 1-28 days,. Animals were sacrificed, hair was clipped short and a strip of dorsal skin was treated with hair remover (Nair). Tape strips were applied sequentially for a total of up to 7 applications from a single position. The strips were washed using a high salt extraction buffer containing 0.5% Triton X-100 and then the keratin was extracted in a buffer containing 1% SDS and 5% 2-mercaptoethanol. Keratin was about 85% pure as determined by gel electrophoresis.

ii). Humans. Healthy human subjects drank 50-100 ml/day of heavy water (deuterated water, ²H₂O) for 1-28 days. Skin strips were applied to the forearm sequentially for up to 20 applications from a single position. The strips were washed and extracted as for the rat strips. Purity of keratin isolated from human skin strips was assessed by gel electrophoresis.

Isolated keratin was further processed and analyzed by GC/MS for deuterium enrichment using techniques known in the art and extensively described in the literature (for a recent example of these techniques, see Fanara et al. (reference 7), supra). For each sample, the excess deuterium incorporated into keratin alanine was determined. Historical data shows that the maximum excess deuterium observed in alanine in animals given 8% ²H₂O is about 0.11.

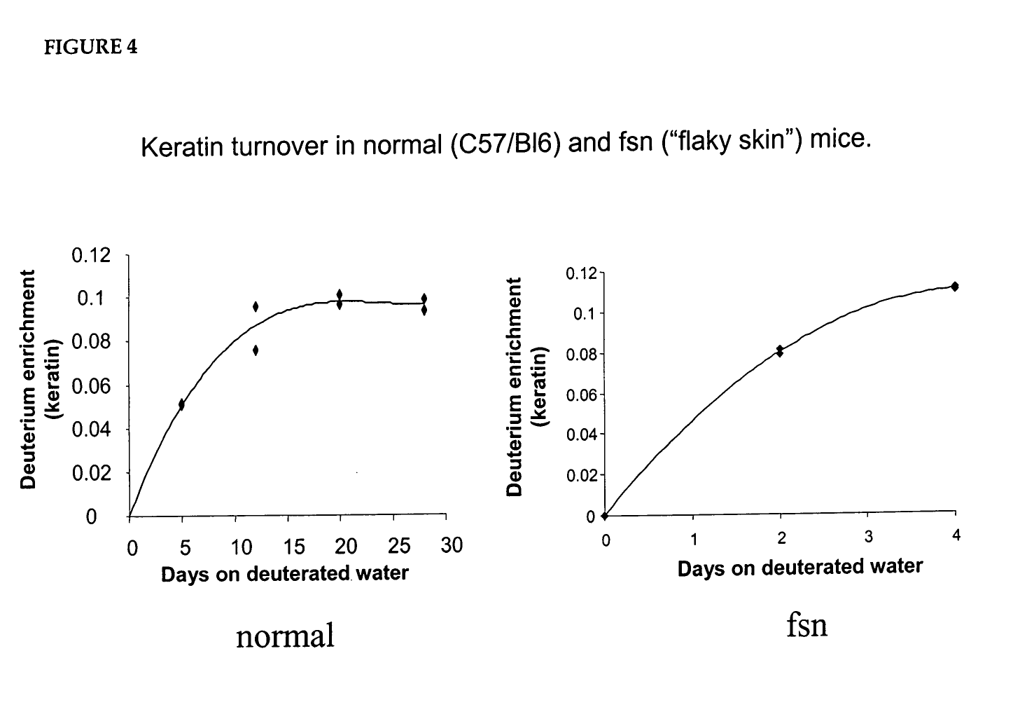

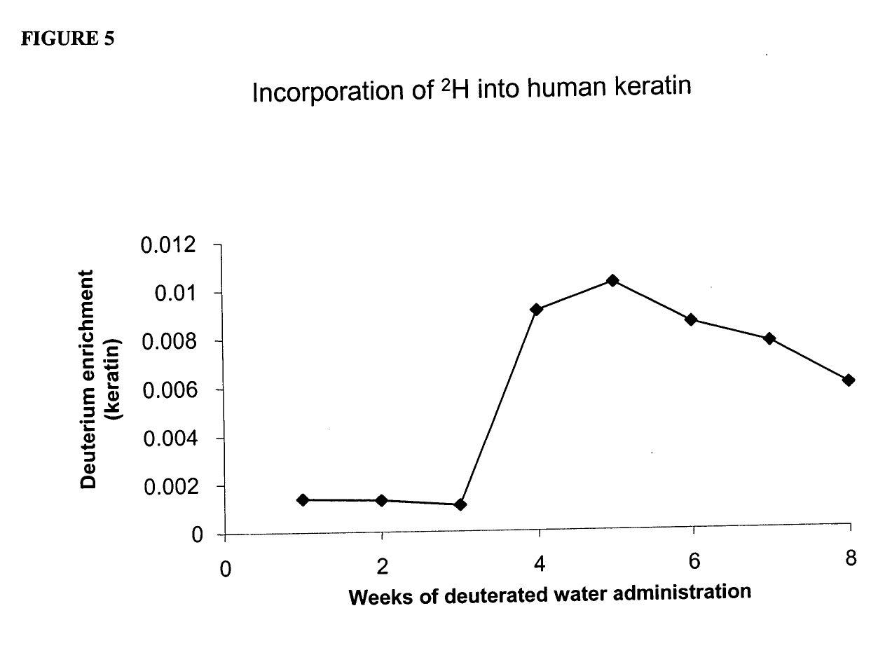

c) results. The results of these experiments are shown in FIG. 4 and FIG. 5. Deuterium incorporation was observed to reach its theoretical maximum in normal mice in 10 to 15 days, and in FSN mice, in about 4 days (FIG. 4). These times represent the time it takes for new keratinocytes to mature and traverse the epithelial layer and arrive at the surface of the skin. In the case of the FSN model of psoriasis, the skin is seen to grow much faster than in normal mice. In humans (FIG. 5), deuterium incorporation is observed beginning between 3 and 4 weeks, and reaches a maximal value at around 5 weeks. In this case, the lag period (weeks 1 to 3) reflects the time it takes for deuterium labeled keratinocytes to reach the accessible outer layers of the skin.

d) discussion. The results of these experiments show that skin proliferation is easily measured using the techniques described here, and that in some models of disease, skin proliferation is dramatically altered with respect to normal. In the case of FSN mice, the hyper-proliferation observed is reflective of the psoriative phenotype. In the case of humans, a similar phenomenon would be expected. Potentially, an animal model like the FSN mouse, or in the clinic, a volunteer with psoriasis, could be given a potential therapeutic for psoriasis and the rate of keratinocyte proliferation could be measured and compared against pre-treatment or no treatment values. In this case, the accelerated proliferation associated with the disease would be expected to be suppressed by an effective treatment.

Alternatively, normal mice or human volunteers could be given potential skin growth treatments or therapeutics and their rate of skin growth could similarly be monitored. Effective treatments or therapeutics would be observed to increase the rate of skin growth, and could be used to treat diabetic ulcers or burns, or could be used for cosmetic purposes.

In either case, the technique gives a quantitative measure of skin proliferation and growth that can be carried out in vivo, and that can be performed over long periods of time in humans without any side effects or discomfort. The range of applications for this particular embodiment of the invention is large, given the number of skin disorders, refractory wound types, and the size of the cosmetic and cosmetic surgery industries.

### Example 4

Isolation of Colon Epithelial Cells from Stool.

a. introduction. Colon cancer is a significant cause of mortality, and other colon disorders, such as irritable bowel disease (IBD) or Crohn's disease, have a large impact on quality of life. Colon cancer is preceded by a hyper-proliferative phase of colon epithelial cell growth (similar to that observed for breast and prostate epithelia, Examples 1 and 2, supra). Crohn's disease or IBD could also include a hyper-proliferative component that exacerbates the symptoms of these unpleasant and painful disorders.

b. methods. Colon epithelial cells are present in the stool, and could be isolated by any number of methods including magnetic or fluorescence activated cell sorting, percoll gradient centrifugation, a number of other mechanical, chemical, enzymatic, or sieving techniques, other techniques known in the art, or a combination of these. Animals or humans can be given ²H₂O by any of the administration protocols listed in the previous examples, supra. Stool can be collected during the course of label administration and colon epithelial cells or other molecules associated of epithelial cell growth (e.g., mucin, a substance secreted into the intestinal lumen by epithelial cells) are isolated from the stool, processed as described supra or by techniques known in the art, and analyzed for deuterium incorporation.

d. discussion. Evaluation of colon epithelial cell proliferation could be an excellent method for evaluating cancer risk, as the hyper-proliferative pre-cancerous promotion phase is well documented, but difficult to study. A subject with or at risk for colon cancer would have increased rates of colon epithelial cell proliferation as compared to a subject not having colon cancer or a subject not having an increased risk of acquiring colon cancer. Similarly, a patient with IBD or Crohn's disease may also have increased proliferation. In these cases, clinical trials using the technique as described could be used to evaluate therapeutics, which in this case would likely result in decreased proliferation if they were effective.

1. A method for evaluating a therapeutic effect of a compound on an epithelial disease of a subject, said method comprising:
a) exposing said subject to one or more compounds; said subject having one or more body spaces wherein said body spaces are in communication with the external environment of said subject;
b) administering an isotope-labeled substrate to said subject for a period of time sufficient for said isotope-labeled substrate to label at least one targeted molecule of interest in one or more metabolic pathways in one or more cells of epithelial origin in said subject;
c) obtaining one or more samples from said one or more body spaces in said subject, wherein said one or more samples comprise at least one isotope-labeled targeted molecule of interest;
d) measuring the content, rate of incorporation and/or pattern or rate of change in content and/or pattern of isotope labeling of said at least one targeted molecule of interest;
e) calculating molecular flux rates in said one or more metabolic pathways of interest based on the content and/or pattern or rate of change of content and/or pattern of isotopic labeling in said at least one targeted molecule of interest;
f) measuring the molecular flux rates in said one or more metabolic pathways of interest according to steps b) through e) in at least one control subject not administered said one or more compounds; and
g) comparing said molecular flux rates in said one or more metabolic pathways of interest in said subject to said molecular flux rates in said one or more metabolic pathways of interest in said control subject not administered said one or more compounds to determine if said one or more compounds has a therapeutic effect on said epithelial disease of said subject.

2. The method of claim 1, wherein the molecular flux rates in said one or more metabolic pathways of interest are relevant to an underlying molecular pathogenesis, or causation of, one or more diseases of epithelial tissue origin.

3. The method of claim 2, wherein the molecular flux rates in said one or more metabolic pathways of interest contribute to the initiation, progression, severity, pathology, aggressiveness, grade, activity, disability, mortality, morbidity, disease sub-classification or other underlying pathogenic or pathologic feature of the one or more diseases of epithelial tissue origin.

4. The method of claim 2, wherein the molecular flux rates in said metabolic pathways of interest contribute to the prognosis, survival, morbidity, mortality, stage, therapeutic response, symptomology, disability or other clinical factor of the one or more diseases of epithelial tissue origin.

5. The method of claim 2, wherein the molecular flux rates of said one or more metabolic pathways of interest are measured concurrently.

6. The method of claim 5, wherein the concurrent measurement of the molecular flux rates from said metabolic pathways of interest is achieved by use of stable isotopic labeling techniques.

7. The method of claim 6, wherein the isotope label used is a stable, non-radioactive isotope.

8. The method of claim 7, wherein the stable isotope used in the stable isotopic labeling is stable isotope-labeled water.

9. The method of claim 8, wherein the stable isotope-labeled water is ²H₂O.

10. The method of claim 5, wherein the concurrent measurement of the molecular flux rates from said metabolic pathways of interest is achieved by use of radioisotope labeling techniques.

11. The method of claim 1, wherein said one or more compound is an already-approved drug.

12. The method of claim 11, wherein the already-approved drug is a Federal Food and Drug Administration-approved drug.

13. The method of claim 11, wherein said already-approved drug is selected randomly.

14. The method of claim 11, wherein said already-approved drug is selected on the basis of a specific biochemical rationale or hypothesis concerning a hypothesized role in the molecular pathogenesis of one or more diseases of epithelial tissue origin.

15. The method of claim 1, wherein said one or more compounds is a new chemical entity.

16. The method of claim 1, wherein said one or more compounds is a biological factor.

17. The method of claim 1, wherein one or more animal models of epithelial tissue disease are used for evaluating said actions on molecular flux rates in one or more metabolic pathways related to an epithelial disease in a subject.

18. The method of claim 17, wherein said one or more animal models of epithelial tissue disease is chosen from psoriasis, skin photoaging, skin rashes, breast cancer, prostate cancer, colon cancer, pancreatic cancer, and lung cancer.

19. The method of claim 1, wherein the one or more metabolic pathways of interest are measured in response to a specific dose or a range of doses of said one or more compounds.

20. The method of claim 1, wherein said one or more metabolic pathways of interest are chosen from breast epithelial cell proliferation, colon epithelial cell proliferation, prostate epithelial cell proliferation, ovarian epithelial cell proliferation, endometrial cell proliferation, bronchial epithelial cell proliferation, pancreatic epithelial cell proliferation, bladder epithelial cell proliferation, keratin synthesis in skin, and keratinocyte proliferation.

21. The method of claim 12, wherein said already-approved drug is screened for actions on multiple biochemical processes concurrently.

22. The method of claim 1, wherein said subject is chosen from rabbits, dogs, mice, rats, guinea pigs, pigs non-human primates, and humans.

23. The method of claim 22, wherein said subject is a human.

24. The method of claim 1, wherein said isotope labeled substrate is chosen from ²H₂O, ²H-glucose, ²H-labeled amino acids, ²H-labeled organic molecules, ¹³C-labeled organic molecules, ¹³CO₂, ¹⁵N-labeled organic molecules, ³H₂O, ³H-labeled glucose, ³H-labeled amino acids, ³H-labeled organic molecules, ¹⁴C-labeled organic molecules, and ¹⁴CO₂.

25. The method of claim 1, wherein said isotope labeled substrate is ²H₂O.

26. The method of claim 1, wherein the one or more compounds are administered according to established or hypothesized dose ranges that have the potential for biological activity in said subject.

27. The method of claim 1, wherein said one or more samples obtained from one or more body spaces in communication with the external environment are collected at known times or intervals after administration or contacting said subject to said isotope-labeled substrate and after exposing said subject to said one or more compound.

28. The method of claim 1, wherein the one or more body spaces in communication with the external environment is chosen from the urethra of the penis, vagina, uterus, gastrointestinal tract, respiratory tract, buccal cavity, skin surface, bladder, and breast duct.

29. The method of claim 1, wherein said one or more samples collected from the one or more body spaces in communication with the external environment is chosen from urine, semen, vaginal secretions, stool, gastrointestinal secretion, sputum, skin flakes, and breast fluid.

30. The method of claim 1, wherein combinations of two or more compounds are exposed to said subject.

31. The method of claim 30, wherein synergistic, complementary, or antagonistic actions of combinations of compounds on molecular flux rates through the one or more metabolic pathways are determined by comparing said molecular flux rates in said subject exposed to the combination of compounds to said molecular flux rates in said subject exposed to a single compound alone or not exposed to any of said compounds being tested.

32. The method of claim 30, wherein said combinations of compounds are selected randomly.

33. The method of claim 30, wherein said combinations of compounds are selected on the basis of a specific biochemical rationale or hypothesis concerning a hypothesized role of one or more of said compounds in the molecular pathogenesis of said one or more of diseases of epithelial tissue origin.

34. A method for evaluating a toxic effect on the epithelial tissue of a subject, said method comprising:
a) exposing said subject to one or more compounds; said subject having one or more body spaces wherein said body sapces are in communication with the external environment of said subject;
b) administering an isotope-labeled substrate to said subject for a period of time sufficient for said isotope-labeled substrate to enter into one or more metabolic pathways of interest and thereby enter into and label one or more targeted molecules of interest within said one or more metabolic pathways of interest in said subject wherein said one or more metabolic pathways of interest are related to at least one biomarker of epithelial tissue toxicity;
c) obtaining one or more samples from said subject, wherein said one or more samples comprise one or more isotope-labeled targeted molecules of interest and are obtained from said one or more body spaces in communication with the external environment;
d) measuring the content, rate of incorporation and/or pattern or rate of change in content and/or pattern of isotope labeling of said targeted molecule or molecules of interest;
e) calculating molecular flux rates in said one or more metabolic pathways of interest based on the content and/or pattern or rate of change of content and/or pattern of isotopic labeling in said molecule or molecules of interest;
f) measuring the molecular flux rates in said one or more metabolic pathways of interest according to steps b) through e) in a control subject not administered said one or more compounds; and
g) comparing said molecular flux rates in said one or more metabolic pathways of interest in said control subject administered said one or more compounds to said molecular flux rates in said one or more metabolic pathways in said subject not administered said one or more compounds to detect any toxic effect to said exposed subject's epithelial tissue.

35. The method of claim 34, wherein said biomarker of epithelial tissue toxicity is chosen from breast epithelial cell proliferation, colon epithelial cell proliferation, prostate epithelial cell proliferation, ovarian epithelial cell proliferation, endometrial cell proliferation, bronchial epithelial cell proliferation, pancreatic epithelial cell proliferation, keratin synthesis in skin, and keratinocyte proliferation.

36. The method of claim 34, wherein the one or more said metabolic pathways of interest related to said biomarker of epithelial tissue toxicity are measured in response to a specific dose or a range of doses of the one or more compounds.

37. The method of claim 34, wherein said subject is chosen from rabbits, dogs, mice, rats, guinea pigs, pigs, and non-human primates.

38. The method of claim 34, wherein said isotope labeled substrate is chosen from ²H₂O, ²H-glucose, ²H-labeled amino acids, ²H-labeled organic molecules, ¹³C-labeled organic molecules, ¹³CO₂, ¹⁵N-labeled organic molecules, ³H₂O, ³H-labeled glucose, ³H-labeled amino acids, ³H-labeled organic molecules, ¹⁴C-labeled organic molecules, and ¹⁴CO₂.

39. The method of claim 34, wherein said isotope labeled substrate is ²H₂O.

40. The method of claim 34, wherein said compounds are administered according to established or hypothesized dose ranges that have the potential for biological activity in said subject.

41. The method of claim 34, wherein said one or more samples obtained from one or more one or more body spaces in communication with the external environment are collected at known times or intervals after administration or contacting said subject to said isotope-labeled substrate and after exposing said subject to said one or more compounds.

42. The method of claim 34, wherein said one or more body spaces in communication with the external environment is chosen from the urethra of the penis, vagina, uterus, gastrointestinal tract, respiratory tract, buccal cavity, skin surface, bladder, and breast duct.

43. The method of claim 34, wherein said one or more samples collected from the one or more body spaces in communication with the external environment is chosen from urine, semen, vaginal secretions, stool, gastrointestinal secretion, sputum, skin flakes, and breast fluid.

44. The method of claim 34, wherein said subject is exposed to combinations of two or more compounds.

45. The method of claim 44, wherein synergistic, complementary, or antagonistic actions of combinations of compounds on molecular flux rates through the one or more metabolic pathways of interest are determined by comparing said molecular flux rates in said subject exposed to the combination of compounds to said molecular flux rates in said subject exposed to a single compound alone or not exposed to any of said one or more compounds being tested.

46. An information storage device comprising data obtained from the method according to claim 1.

47. The device of claim 46, wherein said device is a printed report.

48. The printed report of claim 47, wherein the medium in which said report is printed on is chosen from paper, plastic, and microfiche.

49. The device of claim 46, wherein said device is a computer disc or a computer.

50. The disc of claim 49, wherein said disc is chosen from a compact disc, a digital video disc, an optical disc, and a magnetic disc.

51. An isolated, isotopically-perturbed molecule generated by the method according to claim 1.

52. An isolated isotopically-perturbed molecule generated by the method according to claim 34.

53. The isolated isotopically-perturbed molecule of claim 51, wherein said molecule is chosen from protein, keratin, lipid, nucleic acid, glycosaminoglycan, proteoglycan, porphyrin, and carbohydrate molecules.

54. The isolated isotopically-perturbed molecule of claim 52, wherein said molecule is chosen from protein, keratin, lipid, nucleic acid, glycosaminoglycan, proteoglycan, porphyrin, and carbohydrate molecules.

55. The isolated isotopically-perturbed molecule of claim 51, wherein said molecule is deoxyribonucleic acid or ribonucleic acid.

56. The isolated isotopically-perturbed molecule of claim 52, wherein said molecule is deoxyribonucleic acid or ribonucleic acid.

57. A kit for determining screening of one or more compounds for actions on molecular flux rates in one or more metabolic pathways related to a disease of epithelial tissue origin in a subject, comprising:
a) one or more isotope-labeled precursors, and
b) instructions for use of the kit.

58. The kit of claim 57 further comprising a tool for administration of precursor molecules.

59. The kit of claim 57 further comprising an instrument for collecting a sample from the subject.

60. A kit for determining screening of one or more compounds for actions on molecular flux rates in one or more metabolic pathways related to an effect on one or more biomarkers of toxicity in epithelial tissue of a subject, comprising:
a) one or more isotope-labeled precursors, and
b) instructions for use of the kit.

61. The kit of claim 60 further comprising a tool for administration of precursor molecules.

62. The kit of claim 60 further comprising an instrument for collecting a sample from the subject.

63. The method of claim 1, further comprising the manufacturing of one or more compounds at least partially identified by said method of claim 1.

64. The method of claim 1 further comprising the step of developing one or more compounds at least partially identified by the method of claim 1.

65. The method of claim 64 wherein data from said method are used in said step of developing one or more of said compounds.

66. A method comprising:
a) measuring a molecular flux rate of an epithelial tissue biomarker of interest using an isotope;
b) comparing the results of step a) with a molecular flux rate of an epithelial biomarker of interest in the presence of a compound of interest; and
c) if said compound of interest changes a molecular flux rate of interest, developing said compound as a drug.

67. The method of claim 66, further comprising distributing the therapeutic or diagnostic in commerce.

68. The method of claim 66, further comprising selling the therapeutic or diagnostic.

69. A method for detecting the presence or absence of an epithelial disease or monitoring an epithelial disease of a subject, said method comprising:
a) administering an isotope-labeled substrate to said subject for a period of time sufficient for said isotope-labeled substrate to label at least one targeted molecule of interest in one or more metabolic pathways of interest in one or more cells of epithelial origin in said subject wherein said subject has one or more body spaces wherein said body spaces are in communication with the external environment of said subject;
b) obtaining one or more samples from said one or more body spaces in said subject, wherein said one or more samples comprise at least one isotope-labeled targeted molecule of interest;
c) measuring the content, rate of incorporation and/or pattern or rate of change in content and/or pattern of isotope labeling of said at least one targeted molecule of interest;
d) calculating molecular flux rates in said one or more metabolic pathways of interest based on the content and/or pattern or rate of change of content and/or pattern of isotopic labeling in said at least one targeted molecule of interest; and
e) measuring the molecular flux rates in said one or more metabolic pathways of interest to detect the presence or absence of said epithelial disease or to monitor said epithelial disease in said subject.

70. A method for detecting the presence or absence of cancerous or pre-cancerous breast cancer or monitoring a treatment of breast cancer in a patient, said method comprising:
a) administering deuterated water to said patient for a period of time sufficient for said deuterated water to label DNA in one or more breast epithelial cells in said patient;
b) collecting a sample of said breast epithelial cells comprising said labeled DNA;
c) measuring the content, rate of incorporation and/or pattern or rate of change in content and/or pattern of isotope labeling of said DNA in said breast epithelial cells; and
d) comparing said rate or pattern to historical data, data obtained from a control subject, data obtained from non-cancerous tissue in the same subject, data obtained from the same subject prior to treatment or, or data obtained from the same subject prior to cancer onset in order to evaluate whether or not there is an increase or decrease in breast epithelial cell proliferation with respect to normal subjects or tissues, to detect the presence or absence of cancerous or pre-cancerous breast cancer, or monitor a treatment of breast cancer in said patient.

71. A method for detecting the presence or absence of prostate cancer or monitoring a treatment of prostate cancer in a patient, said method comprising:
a) administering deuterated water to said patient for a period of time sufficient for said deuterated water to label DNA in one or more prostate epithelial cells in said patient;
b) collecting a sample of said prostate epithelial cells comprising said labeled DNA;
c) measuring the content, rate of incorporation and/or pattern or rate of change in content and/or pattern of isotope labeling of said DNA in said prostate epithelial cells; and
d) comparing said rate or pattern to historical data, data obtained from a control subject, data obtained from tissue in the same subject, data obtained from the same subject prior to treatment, or data obtained from the same subject prior to cancer onset in order to evaluate whether or not there is an increase or decrease in prostate epithelial cell proliferation with respect to normal subjects or tissues, to detect the presence or absence of prostate cancer, or monitor treatment of prostate cancer in said patient.

72. A method for detecting the presence or absence of a skin disorder or monitoring a treatment of a skin disorder in a patient, said method comprising:
a) administering deuterated water to said patient for a period of time sufficient for said deuterated water to label keratin in the skin of said patient;
b) collecting a sample of said labeled keratin;
c) measuring the content, rate of incorporation and/or pattern or rate of change in content and/or pattern of isotope labeling of said keratin; and
d) comparing said rate or pattern to historical data, data obtained from a control subject, data obtained from non-disordered tissue in the same subject, data obtained from the same subject prior to treatment, or data obtained from the same subject prior to disorder onset in order to evaluate whether or not there is an increase or decrease in keratin synthesis with respect to normal subjects or tissues, to detect the presence or absence of a skin disorder or to monitor the treatment of a skin disorder.

73. The method of claim 72 wherein said skin disorder is psoriasis or eczema.

74. A method for detecting the presence or absence of a colon disorder or monitoring a treatment of a colon disorder in a patient, said method comprising:
a) administering deuterated water to said patient for a period of time sufficient for said deuterated water to label mucin in one or more colon epithelial cells in said patient;
b) collecting a sample of stool or epithelial tissue or colon epithelial cells comprising said labeled mucin;
c) measuring the content, rate of incorporation and/or pattern or rate of change in content and/or pattern of isotope labeling of said mucin in said colon epithelial cells;
d) comparing said rate or pattern to historical data, data obtained from a control subject, data obtained from non-disorder tissue in the same subject, data obtained from the same subject prior to treatment, or data obtained from the same subject prior to disorder onset in order to evaluate whether or not there is an increase or decrease in mucin synthesis with respect to normal subjects or tissues, to detect the presence or absence of a colon disorder or monitor the treatment of a colon disorder.

75. The method of claim 74 wherein said colon disorder is colon cancer, irritable bowl disease or Crohn's disease.

76. A method for detecting the presence or absence of a colon disorder or monitoring a treatment of a colon disorder in a patient, said method comprising:
a) administering deuterated water to said patient for a period of time sufficient for said deuterated water to label DNA in one or more colon epithelial cells in said patient thereby forming labeled colon epithelial cell DNA;
b) collecting a sample of stool or tissue comprising said labeled colon epithelial cell DNA;
c) measuring the content, rate of incorporation and/or pattern or rate of change in content and/or pattern of isotope labeling of said DNA in said colon epithelial cells;
d) comparing said rate or pattern to historical data, data obtained from a control subject, data obtained from non-disorder tissue in the same subject, data obtained from the same subject prior to treatment, or data obtained from the same subject prior to disorder onset in order to evaluate whether or not there is an increase or decrease in colon epithelial cell proliferation with respect to normal subjects or tissues, to detect the presence or absence of a colon disorder or monitor the treatment of a colon disorder.

77. The method of claim 76 wherein said colon disorder is colon cancer, irritable bowl disease or Crohn's disease.
